SEQUENCE LISTING (47 sequences)

SEQ ID NO: 1 - protein, 8 aa, synthetic construct
GYTFTSYY

SEQ ID NO: 2 - protein, 8 aa, synthetic construct
IWPGDGST

SEQ ID NO: 3 - protein, 13 aa, synthetic construct
ASPMITPNYAMDY

SEQ ID NO: 4 - protein, 6 aa, synthetic construct
QDISKY

SEQ ID NO: 5 - skipped

SEQ ID NO: 6 - protein, 9 aa, synthetic construct
QQGSTLPYT

SEQ ID NO: 7 - protein, 120 aa, synthetic construct
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSS

SEQ ID NO: 8 - protein, 107 aa, synthetic construct
DIQMTQSPSSLSASVGDRVTITCRASQDISKYLNWYQQKPGKAPKLLIYYTSRLQSGVPSRFSGRGSGTDYSLTISSLQPEDFATYFCQQGSTLPYTFGGGTKVEIK

SEQ ID NO: 9 - protein, 449 aa, synthetic construct
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
DIQMTQSPSSLSASVGDRVTITCRASQDISKYLNWYQQKPGKAPKLLIYYTSRLQSGVPSRFSGRGSGTDYSLTISSLQPEDFATYFCQQGSTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 450 aa, synthetic construct
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 450 aa, synthetic construct
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGH

SEQ ID NO: 13 - protein, 450 aa, synthetic construct
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGR

SEQ ID NO: 14 - protein, 450 aa, synthetic construct
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGD

SEQ ID NO: 15 - protein, 450 aa, synthetic construct
QVQLVQSGAEVKKPGASVKVSCKASGYTFTSYYIHWVRQAPGQGLEWMGWIWPGDGSTKYAQKFQGRVTMTRDTSTSTVYMELSSLRSEDTAVYFCASPMITPNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGE

SEQ ID NO: 16 - protein, 21 aa, synthetic construct
QAPGQGLEWMGWIWPGDGSTK

SEQ ID NO: 17 - protein, 37 aa, synthetic construct
SEDTAVYFCASPMITPNYAMDYWGQGTLVTVSSASTK

SEQ ID NO: 18 - protein, 63 aa, synthetic construct
DYFPEPVTVSWNSGALTSGVHTFPAVLQSSGLYSLSSVVTVPSSSLGTQTYICNVNHKPSNTK

SEQ ID NO: 19 - protein, 14 aa, synthetic construct
FNWYVDGVEVHNAK

SEQ ID NO: 20 - protein, 9 aa, synthetic construct
EEQYNSTYR

SEQ ID NO: 21 - protein, 16 aa, synthetic construct
VVSVLTVLHQDWLNGK

SEQ ID NO: 22 - protein, 11 aa, synthetic construct
EPQVYTLPPSR

SEQ ID NO: 23 - protein, 10 aa, synthetic construct
NQVSLTCLVK

SEQ ID NO: 24 - protein, 22 aa, synthetic construct
GFYPSDIAVEWESNGQPENNYK

SEQ ID NO: 25 - protein, 23 aa, synthetic construct
WQQGNVFSCSVMHEALHNHYTQK

SEQ ID NO: 26 - protein, 18 aa, synthetic construct
DIQMTQSPSSLSASVGDR

SEQ ID NO: 27 - protein, 11 aa, synthetic construct
YLNWYQQKPGK

SEQ ID NO: 28 - protein, 8 aa, synthetic construct
LQSGVPSR

SEQ ID NO: 29 - protein, 38 aa, synthetic construct
GSGTDYSLTISSLQPEDFATYFCQQGSTLPYTFGGGTK

SEQ ID NO: 30 - protein, 38 aa, synthetic construct; identical to SEQ ID NO: 29

SEQ ID NO: 31 - protein, 16 aa, synthetic construct
SGTASVVCLLNNFYPR

SEQ ID NO: 32 - protein, 4 aa, synthetic construct
VQWK

SEQ ID NO: 33 - protein, 20 aa, synthetic construct
VDNALQSGNSQESVTEQDSK

SEQ ID NO: 34 - protein, 17 aa, synthetic construct
VYACEVTHQGLSSPVTK

SEQ ID NO: 35 - protein, 21 aa, synthetic construct
QAPGQGLEWMGWIWPGDGSTK

SEQ ID NO: 36 - protein, 5 aa, synthetic construct
VTMTR

SEQ ID NO: 37 - protein, 15 aa, synthetic construct
DTSTSTVYMELSSLR

SEQ ID NO: 38 - protein, 37 aa, synthetic construct; identical to SEQ ID NO: 17

SEQ ID NO: 39 - protein, 63 aa, synthetic construct; identical to SEQ ID NO: 18

SEQ ID NO: 40 - protein, 7 aa, synthetic construct
DTLMISR

SEQ ID NO: 41 - protein, 14 aa, synthetic construct
FNWYVDGVEVHNAK

SEQ ID NO: 42 - protein, 16 aa, synthetic construct
VVSVLTVLHQDWLNGK

SEQ ID NO: 43 - protein, 22 aa, synthetic construct
GFYPSDIAVEWESNGQPENNYK

SEQ ID NO: 44 - protein, 23 aa, synthetic construct
WQQGNVFSCSVMHEALHNHYTQK

SEQ ID NO: 45 - protein, 18 aa, synthetic construct
DIQMTQSPSSLSASVGDR

SEQ ID NO: 46 - protein, 11 aa, synthetic construct
YLNWYQQKPGK

SEQ ID NO: 47 - protein, 4 aa, synthetic construct
VQWK

(19) United States
(12) Patent Application Publication
(10) Pub. No.: US 2026/0070991 A1
(43) Pub. Date: Mar. 12, 2026

(54) ANTI-IGF-1R ANTIBODY COMPOSITIONS

(71) Applicants: ACELYRIN, INC., Agoura Hills, CA (US); PIERRE FABRE MEDICAMENT SAS, Lavaur (FR)

(72) Inventors: Tatyana Touzova, Los Angeles, CA (US); Anita Grover, Los Angeles, CA (US); Andrew Nyborg, Los Angeles, CA (US); Paul Peloso, Agoura Hills, CA (US); Shao-Lee Lin, Agoura Hills, CA (US)

(21) Appl. No.: 18/872,687

(22) PCT Filed: Jun. 9, 2023
(86) PCT No.: PCT/US2023/068179

§ 371 Date: Dec. 6, 2024

Related U.S. Application Data
(60) Provisional application No. 63/351,120, filed on Jun. 10, 2022.
(60) Provisional application No. 63/409,301, filed on Sep. 23, 2022.
(60) Provisional application No. 63/450,523, filed on Mar. 7, 2023.

(51) Int. Cl. C07K 16/28; A61K 9/00; A61K 9/08; A61K 39/00; A61K 47/22; A61K 47/26; A61M 5/20; A61P 27/02

(52) U.S. Cl. CPC C07K 16/2863; A61K 9/0019; A61K 9/08; A61K 47/22; A61K 47/26; A61M 5/20; A61P 27/02; A61K 2039/505; A61K 2039/54; A61K 2039/545; A61M 2005/206; A61M 2210/0612

(57) ABSTRACT

The present disclosure is generally directed to pharmaceutical compositions comprising anti-IGF-IR antibodies and methods of making and using such compositions. In certain embodiments, such compositions exhibit low viscosity and high stability and can therefore be conveniently delivered to subjects in need thereof with reduced discomfort. Also provided herein are anti-IGF-IR antibodies that have a heavy chain comprising charged amino acid on its c-terminus.

### BACKGROUND

Thyroid eye disease (TED), also known as Graves' ophthalmopathy, is an autoimmune inflammatory disorder of the orbit and periorbital tissues, characterized by upper eyelid retraction, lid lag, swelling, redness (erythema), conjunctivitis, and bulging eyes (exophthalmos). It occurs most commonly in individuals with Graves' disease, and less commonly in individuals with Hashimoto's thyroiditis, or in those who are euthyroid. It is part of a systemic process with variable expression in the eyes, thyroid, and skin, caused by autoantibodies that bind to tissues in those organs. The autoantibodies target the fibroblasts in the eye muscles, and those fibroblasts can differentiate into fat cells (adipocytes). Fat cells and muscles expand and become inflamed. Veins become compressed and are unable to drain fluid, causing edema.

Current therapies for hyperthyroidism due to Graves' disease are imperfect because therapies targeting the specific underlying pathogenic autoimmune mechanisms of the disease are lacking. Even more complex is the treatment of moderate-to-severe active TED. Although recent years have witnessed a better understanding of its pathogenesis, TED remains a therapeutic challenge and dilemma. Accordingly, there is a significant unmet need for treatments for patients with TED and its related symptoms.

### SUMMARY

There is an unmet need for development of therapies for treatment of TED with greater depth and durability of response. Additionally, the currently available therapies for treatment of TED have side-effects, including hearing impairment. These side-effects, which are reported in a substantial proportion of patients receiving currently available therapy, have limited treatment in patients with TED.

In certain aspects, provided herein are pharmaceutical compositions comprising anti-IGF-IR antibodies, as well as methods of using and making such pharmaceutical compositions. In certain embodiments, the pharmaceutical compositions of the invention are IGF-1R antibody solutions. In certain embodiments, the pharmaceutical compositions provided herein comprise a high-concentration of anti-IGF-1R antibody solution (e.g., such as higher than 75 mg/ml or higher than 100 mg/ml) while exhibiting a high agent stability and low viscosity. Such compositions are therefore amenable to subcutaneous delivery, including self-delivery, to patients suffering from thyroid eye disease, as well as other diseases and disorders for which anti-IGF-1R antibodies may provide a therapeutic benefit. Moreover, the high antibody concentration coupled with low viscosity of compositions provided herein allow for delivery to patients of lower volumes of composition through smaller needles, reducing patient discomfort and further facilitating self-delivery.

In certain aspects, provided herein is a pharmaceutical composition comprising: (a) at least 75 mg/ml of an anti-IGF-1R antibody; (b) from 20 to 30 mM histidine; and (c) from 4% to 6% of a sugar; wherein the pharmaceutical composition is in the pH range of 5.5-6.5. In some embodiments, the sugar is D-sorbitol.

In certain embodiments, the anti-IGF-IR antibody comprises a CDRH1 of SEQ ID NO: 1, a CDRH2 of SEQ ID NO: 2, a CDRH3 of SEQ ID NO: 3, a CDRL1 of SEQ ID NO: 4, a CDRL2 of SEQ ID NO: 5, and a CDRL3 of SEQ ID NO: 6. In some embodiments, the antibody comprises a heavy chain variable domain comprising an amino acid sequence of SEQ ID NO: 7. In some embodiments, the antibody comprises a light chain variable domain comprising an amino acid sequence of SEQ ID NO: 8. In certain embodiments, the antibody comprises a heavy chain comprising an amino acid sequence of SEQ ID NO: 9. In some embodiments, the antibody comprises a light chain comprising an amino acid sequence of SEQ ID NO: 10. In some embodiments, the antibody comprises a heavy chain further comprising a charged amino acid at its c-terminus (e.g., a c-terminal arginine, histidine, lysine, aspartic acid, or glutamic acid). In some embodiments, the charged amino acid is a positively charged amino acid (e.g., arginine, histidine, or lysine). In some embodiments the charged amino acid is a negatively charged amino acid (e.g., aspartic acid, or glutamic acid). In some embodiments, the antibody comprises a heavy chain comprising an amino acid sequence of SEQ ID NO: 11, 12, 13, 14, or 15. In some embodiments, the antibody comprises a human IgG1 heavy chain constant domain and a human kappa light chain constant domain. In certain embodiments, the anti-IGF-1R antibody is lonigutamab.

Lonigutamab is a humanized monoclonal antibody against IGF-1R. Lonigutamab has high affinity and specificity toward IGF-IR. Specifically, lonigutamab has picomolar affinity for IGF-1R. In certain preferred embodiments, lonigutamab has a k_{D} of about 30 PM to a binding epitope of IGF-IR. Once lonigutamab binds to IGF-1R, it induces receptor internalization, which results in signal blockade from IGF-IR, which may lead to the therapeutic effect of lonigutamab. The IGF-IR internalization may occur within minutes after administration of pharmaceutical compositions comprising lonigutamab. In certain preferred embodiments, the therapeutically effective serum concentration of lonigutamab may be attained in about an hour after administration of the pharmaceutical composition comprising lonigutamab to a patient.

Lonigutamab has unique and beneficial pharmacological properties. In certain preferred embodiments, administration of pharmaceutical compositions comprising lonigutamab induce greater than about 70% IGF-IR internalization. In certain preferred embodiments, administration of pharmaceutical compositions comprising lonigutamab induce greater than about 80% IGF-IR internalization. In certain preferred embodiments, administration of pharmaceutical compositions comprising lonigutamab induce greater than about 85% IGF-IR internalization. In certain preferred embodiments, administration of pharmaceutical compositions comprising lonigutamab induce greater than about 90% IGF-1R internalization.

In certain embodiments, the pharmaceutical composition comprises at least 75 mg/ml of anti-IGF-IR antibody. In some embodiments, the pharmaceutical composition comprises at least 100 mg/ml, at least 125 mg/ml, at least 150 mg/ml, at least 175 mg/ml, at least 200 mg/ml, or at least 250 mg/ml of the anti-IGF-1R antibody. In certain embodiments, the pharmaceutical composition comprises from 75 mg/ml to 300 mg/ml, from 100 mg/ml to 300 mg/ml, or from 125 mg/ml to 250 mg/ml of the anti-IGF-1R antibody. In some embodiments, the pharmaceutical composition comprises about 125 mg/ml, about 150 mg/ml, about 175 mg/ml, about 200 mg/ml, or about 250 mg/ml of the anti-IGF-1R antibody.

In some embodiments, the pharmaceutical composition comprises from 20 to 30 mM histidine. In certain embodiments, the pharmaceutical composition comprises about 20 mM, about 21 mM, about 22 mM, about 23 mM, about 24 mM, about 25 mM, about 26 mM, about 27 mM, about 28 mM, about 29 mM, or about 30 mM histidine.

In some embodiments, the pharmaceutical composition comprises from 4% to 6% of a sugar (e.g., D-sorbitol). In certain embodiments, the pharmaceutical composition comprises about 4%, 5%, or 6% of a sugar (e.g., D-sorbitol).

In some embodiments, the pharmaceutical composition is at a pH of from 5.5 to 6.5. In certain embodiments, the pharmaceutical composition is at a pH of about 5.5, about 5.6, about 5.7, about 5.8, about 5.9, about 6.0, about 6.1, about 6.2, about 6.3, about 6.4, or about 6.5.

In some embodiments, the pharmaceutical composition does not comprise polysorbate 80. In some embodiments, the pharmaceutical composition comprises a small amount of polysorbate 80 (e.g., less than 0.05%). In certain embodiments, the pharmaceutical composition comprises no more than 0.05% polysorbate 80. In some embodiments, the pharmaceutical composition comprises no more than 0.02% polysorbate 80. In some embodiments, the pharmaceutical composition comprises no more than 0.01% polysorbate 80. In some embodiments, the pharmaceutical composition comprises no more than 0.005% polysorbate 80. In certain embodiments, the pharmaceutical composition comprises from 0.002% to 0.05% polysorbate 80. In some embodiments, the pharmaceutical composition comprises about 0.002%, about 0.003%, about 0.004%, about 0.005%, about 0.006%, about 0.007%, about 0.008%, about 0.009%, about 0.01%, about 0.015%, about 0.02%, about 0.025%, about 0.03%, about 0.035%, about 0.04%, about 0.045%, or about 0.05% polysorbate 80.

In some embodiments, the pharmaceutical composition does not comprise poloxamer 188 (P188). In some embodiments, the pharmaceutical composition comprises a small amount of poloxamer 188 (e.g., less than 0.1%). In certain embodiments, the pharmaceutical composition comprises no more than 0.1% poloxamer 188. In some embodiments, the pharmaceutical composition comprises no more than 0.05% poloxamer 188. In some embodiments, the pharmaceutical composition comprises no more than 0.02% poloxamer 188. In some embodiments, the pharmaceutical composition comprises no more than 0.01% poloxamer 188. In some embodiments, the pharmaceutical composition further comprises from 0.01% to 0.1% poloxamer 188. In certain embodiments, the pharmaceutical composition comprises about 0.01%, about 0.02%, about 0.03%, about 0.04%, about 0.05%, about 0.06%, about 0.07%, about 0.08%, about 0.09%, or about 0.1% poloxamer 188.

In certain embodiments, the osmolality of the pharmaceutical composition is within physiological osmolality range of 250-400 mOsm/kg.

In certain embodiments, the viscosity of the pharmaceutical composition is no more than 30 cP at 21° C. In certain embodiments, the viscosity of the pharmaceutical composition is no more than 15 cP at 21° C. In certain embodiments, the viscosity of the pharmaceutical composition is about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, about 23 cP, about 24 cP, about 25 cP, about 26 cP, about 27 cP, about 28 cP, about 29 cP, or about 30 cP at 21° C.

In certain embodiments, the pharmaceutical composition is stable for at least 8 weeks, at least 9 weeks, at least 10 weeks, at least 11 weeks, at least 12 weeks, at least 13 weeks, at least 14 weeks, at least 15 weeks, at least 16 weeks, or longer. In some embodiments, the pharmaceutical composition is stable for such a length of time stable at a temperature of from −70° C. to 8° C. (e.g., at about −70° C., at about −20° C., at about 4° C.).

In some aspects, provided herein is an injector or a syringe comprising the pharmaceutical composition provided herein. In some embodiments, the injector comprises a delivery volume of no more than 2 ml (e.g., no more than 1.5 ml, no more than 1 ml). In some embodiments, the injector comprises a needle of a size of no bigger than 24G (e.g., 25G, 26G). In certain embodiments, the injector is an automatic reusable fix dose device. In some embodiments, the injector is an automatic reusable variable dose device. In some embodiments, the injector is an automatic disposable fix dose autoinjector.

In certain aspects, provided herein is an anti-IGF-1R antibody comprises a CDRH1 of SEQ ID NO: 1, a CDRH2 of SEQ ID NO: 2, a CDRH3 of SEQ ID NO: 3, a CDRL1 of SEQ ID NO: 4, a CDRL2 of SEQ ID NO: 5, and a CDRL3 of SEQ ID NO: 6, wherein the heavy chain of the antibody comprises a charged amino acid at its c-terminus (e.g., a c-terminal arginine, histidine, lysine, aspartic acid, or glutamic acid). In some embodiments, the charged amino acid is a positively charged amino acid (e.g., arginine, histidine, or lysine). In some embodiments the charged amino acid is a negatively charged amino acid (e.g., aspartic acid, or glutamic acid). In some embodiments, the antibody comprises a heavy chain variable domain comprising an amino acid sequence of SEQ ID NO: 7. In some embodiments, the antibody comprises a light chain variable domain comprising an amino acid sequence of SEQ ID NO: 8. In some embodiments, the antibody comprises a light chain comprising an amino acid sequence of SEQ ID NO: 10. In some embodiments, the antibody comprises a heavy chain comprising an amino acid sequence of SEQ ID NO: 11, 12, 13, 14, or 15. In some embodiments, provided herein is a pharmaceutical composition comprising the anti-IGF-1R antibody.

In certain aspects, provided herein are methods of treating a disease or disorder comprising administering a pharmaceutical composition and/or an antibody provided herein. In some embodiments, the pharmaceutical composition is administered using an injector provided herein. In certain embodiments, the disease or disorder is thyroid eye disease (TED). In some embodiments, the disease or disorder is stroke, acromegaly, diabetic nephropathy (diabetic kidney disease), idiopathic pulmonary fibrosis, interstitial lung disease, obesity, type 2 diabetes, juvenile idiopathic arthritis (JIA), diffuse cutaneous systemic sclerosis, Sjogren's syndrome calcinosis and vasculitis, cachexia and sarcopenia, diabetic macular edema, arterial atherosclerosis, peripheral artery disease (PAD), myocardial infarction and stroke, diabetic foot and skin, rheumatoid arthritis, neurofibromatosis 1, neurofibromatosis 2, polycystic kidney disease, polycystic liver disease, polycystic ovarian syndrome, Alzheimers disease, cognitive decline, dementia, depression and anxiety, asthma, aging, thyroid eye disease, idiopathic orbital inflammation, human type 2 lipodystrophy and associated cardiomyopathy, autosomal dominant polycystic kidney disease (ADPKD), NASH, Graves disease, and/or Hashimoto's thyroiditis.

In certain embodiments, the pharmaceutical composition and/or antibody provided herein is for use in treating a disease or disorder. In some embodiments, the disease or disorder is thyroid eye disease (TED). In some embodiments, the disease or disorder is stroke, acromegaly, diabetic nephropathy (diabetic kidney disease), idiopathic pulmonary fibrosis, interstitial lung disease, obesity, type 2 diabetes, juvenile idiopathic arthritis (JIA), diffuse cutaneous systemic sclerosis, Sjogren's syndrome calcinosis and vasculitis, cachexia and sarcopenia, diabetic macular edema, arterial atherosclerosis, peripheral artery disease (PAD), myocardial infarction and stroke, diabetic foot and skin, rheumatoid arthritis, neurofibromatosis 1, neurofibromatosis 2, polycystic kidney disease, polycystic liver disease, polycystic ovarian syndrome, Alzheimers disease, cognitive decline, dementia, depression and anxiety, asthma, aging, thyroid eye disease, idiopathic orbital inflammation, human type 2 lipodystrophy and associated cardiomyopathy, autosomal dominant polycystic kidney disease (ADPKD), NASH, Graves disease, and/or Hashimoto's thyroiditis.

In certain embodiments of the compositions and methods provided herein, the pharmaceutical composition is formulated for subcutaneous administration. In some embodiments, the pharmaceutical composition is administered subcutaneously. In certain embodiments, the pharmaceutical composition is formulated for intramuscular administration. In certain embodiments, the pharmaceutical composition is administered intramuscularly. In some embodiments, the pharmaceutical composition is formulated for infraorbital, intravitreal, intraocular, subconjunctival, retrobulbar, peribulbar, and/or intrathecal administration. In certain embodiments, the pharmaceutical composition is administered by infraorbital, intravitreal, intraocular, subconjunctival, retrobulbar, peribulbar, and/or intrathecal injection.

In certain embodiments, the pharmaceutical composition is administered in a delivery volume of no more than 3 ml, 2.5 ml, 2 ml, 1.5 ml, or 1 ml. In some embodiments, the pharmaceutical composition is administered in a delivery volume of no more than 2 ml.

In certain embodiments, the pharmaceutical composition is administered via a needle of a size of no bigger than 24G, 25G, or 27G. In some embodiments, the pharmaceutical composition is administered in a 24G needle, a 25G needle, or a 27G needle.

In certain embodiments, the pharmaceutical composition is administered with an injection force of no more than 14N, 13N, 12N, 11N, 10N, 9N, 8N, 7N, or 6N. In some embodiments, the pharmaceutical composition is administered with an injection force of no more than 12N. In certain embodiments, the pharmaceutical composition is administered with an injection force of about 4N, about 5N, about 6N, about 7N, about 8N, about 9N, about 10N, about 11N, or about 12N.

In certain embodiments, the method provided herein treats and/or prevents one or more disease or disorder. In some embodiments, the disease or disorder is thyroid eye disease (TED). In some embodiments, the disease or disorder is stroke, acromegaly, diabetic nephropathy (diabetic kidney disease), idiopathic pulmonary fibrosis, interstitial lung disease, obesity, type 2 diabetes, juvenile idiopathic arthritis (JIA), diffuse cutaneous systemic sclerosis, Sjogren's syndrome calcinosis and vasculitis, cachexia and sarcopenia, diabetic macular edema, arterial atherosclerosis, peripheral artery disease (PAD), myocardial infarction and stroke, diabetic foot and skin, rheumatoid arthritis, neurofibromatosis 1, neurofibromatosis 2, polycystic kidney disease, polycystic liver disease, polycystic ovarian syndrome, Alzheimers disease, cognitive decline, dementia, depression and anxiety, asthma, aging, thyroid eye disease, idiopathic orbital inflammation, human type 2 lipodystrophy and associated cardiomyopathy, autosomal dominant polycystic kidney disease (ADPKD), NASH, Graves disease, and/or Hashimoto's thyroiditis.

In certain embodiments, the method reduces the severity of the thyroid eye disease (TED). In the method reduces proptosis in an eye in a subject with thyroid eye disease (TED). In some embodiments, proptosis is reduced by at least 2 mm, at least 3 mm, or at least 4 mm.

In some embodiments, the method provided herein reduces Clinical Activity Score (CAS) of thyroid eye disease (TED). In some embodiments, the clinical activity score (CAS) is reduced by at least 2 points. In certain embodiments, the clinical activity score (CAS) is reduced to one (1). In certain embodiments, the clinical activity score (CAS) of the subject is reduced to zero (0).

In some embodiments, the method provided herein improves the European Group on Graves' Orbitopathy Clinical Activity Score (EUGOGO CAS) of the subject (e.g., by at least 1, 2, or 3). In certain embodiments, the method improves the pa's Clinical Measures of Severity Score (CMSS) (e.g., by at least 1, 2, or 3).

In certain embodiments, the method provided herein improves the quality of life in the subject. In some embodiments, the quality of life is measured by the Graves' Ophthalmopathy Quality of Life (GO-QoL) assessment. In certain embodiments, the quality of life is measured by the Visual Functioning or Appearance subscale thereof. In some embodiments, the quality of life is measured by the European Group on Graves' orbitopathy (EUGOGO) guidelines. In certain embodiments, the method provided herein reduces the severity of diplopia (e.g., constant diplopia, inconstant diplopia, intermittent diplopia).

In certain aspects, the invention provides methods of administration of pharmaceutical compositions comprising lonigutamab. These pharmaceutical compositions may be administered to patients suffering from thyroid eye disease (TED). In certain embodiments, the pharmaceutical compositions of the invention may be administered intravenously. The methods of the invention provide intravenous infusion of pharmaceutical compositions comprising up to about 3.0 mg/kg lonigutamab to the patient. In certain embodiments, methods of the invention provide administration of pharmaceutical compositions comprising about 0.1, 0.3, 1.0, or 3.0 mg/kg of lonigutamab as an intravenous infusion. The invention further provides that the intravenous infusion may take place for a period of about 15 minutes to about 120 minutes. In certain preferred embodiments, the intravenous infusion takes place for a duration of about 60 minutes.

In certain aspects, the invention provides administration of pharmaceutical compositions comprising lonigutamab as a subcutaneous injection. In certain embodiments, the methods of the invention provide subcutaneous administration of up to about 250 mg lonigutamab. In certain embodiments, the pharmaceutical composition for subcutaneous administration comprises about 20 mg, 40 mg, 125 mg, or 250 mg of lonigutamab. The pharmaceutical compositions for subcutaneous administration may have a volume of from about 0.1 mL to about 3 mL. In certain embodiments, the pharmaceutical compositions for subcutaneous administration may have a volume of from about 0.5 mL to about 3 mL. In preferred embodiments, the administered volume is up to about 2 mL. Beneficially, since the subcutaneous administration is conducted within tolerable volumes, the pharmaceutical compositions could be administered in an out-patient setting, or the pharmaceutical compositions of the invention could be self-administered by the patients. This results in a convenient and an efficient way for administration of the pharmaceutical composition comprising lonigutamab. In particular, the subcutaneous administration of pharmaceutical compositions is beneficial as compared to the intravenous administration due to the ease of administration of the pharmaceutical composition. Moreover, as demonstrated by the pharmacokinetic data provided herein, the subcutaneous administration of pharmaceutical compositions comprising lonigutamab results in a advantageous pharmacokinetic and pharmacodynamic characteristic of the lonigutamab. Specifically, the subcutaneous administration of pharmaceutical compositions comprising lonigutamab results in a higher therapeutically effective serum concentration of lonigutamab for a longer duration.

In certain embodiments of the invention, the administration of pharmaceutical composition comprising lonigutamab results in maximal IGF-IR receptor occupancy with the anti-IGF-1R antibody. This maximal occupancy of IGF-IR may be achieved any time after the administration of the pharmaceutical composition comprising lonigutamab. In certain embodiments, this is achieved after about 12 hours after administration of the pharmaceutical composition comprising lonigutamab. In certain embodiments, the pharmaceutical compositions administered may comprise from about 10 mg to about 500 mg lonigutamab. In certain embodiments, the pharmaceutical compositions administered may comprise from about 20 mg to about 400 mg lonigutamab. In preferred embodiments, the pharmaceutical compositions administered may comprise about 100 mg to about 300 mg lonigutamab. In preferred embodiments, the pharmaceutical compositions administered may comprise about 20 mg or about 40 mg lonigutamab. In preferred embodiments, the pharmaceutical composition may comprise about 125 mg or about 250 mg of lonigutamab. Notably, the maximal IGF-1R receptor occupancy may be maintained up to a few months after a single administration. In preferred embodiments, the IGF-IR receptor occupancy maintained for at least about 4 weeks after a single administration of a pharmaceutical composition comprising lonigutamab. The maximal receptor occupancy level may be maintained after a single subcutaneous injection or an intravenous infusion of pharmaceutical composition comprising lonigutamab.

In certain aspects, the serum concentration required for maximal IGF-1R receptor internalization is a concentration of at least about 3 μg/mL of lonigutamab. Thus, the therapeutically effective serum concentration of lonigutamab is at least about 1 μg/mL. In certain preferred embodiments, the therapeutically effective serum concentration of lonigutamab is at least about 3 μg/mL. Importantly, the pharmaceutical compositions of the invention provide a therapeutically effective serum concentration of lonigutamab to the patient.

In certain aspects of the invention, the pharmaceutical compositions comprising lonigutamab administered to the patients are safe and tolerable. Moreover, the pharmaceutical compositions of the invention have minimal adverse events.

In certain aspects, the invention provides methods of treatment of thyroid eye disease (TED) comprising subcutaneous administration of a pharmaceutical composition comprising from about 10 mg to about 500 mg lonigutamab. In certain embodiments, the pharmaceutical composition comprises about 20 mg lonigutamab. In certain embodiments, the pharmaceutical composition comprises about 40 mg lonigutamab. In certain embodiments, the pharmaceutical composition comprises about 125 mg lonigutamab. In certain embodiments, the pharmaceutical composition comprises about 250 mg lonigutamab.

In certain embodiments, the pharmaceutical composition comprising lonigutamab is administered once weekly. In certain embodiments, the pharmaceutical composition comprising lonigutamab is administered twice weekly. In certain embodiments, the pharmaceutical composition comprising lonigutamab is administered thrice weekly. In certain embodiments, the pharmaceutical composition comprising lonigutamab is administered on alternate days, i.e., once every two days. In certain preferred embodiments, the pharmaceutical composition comprising lonigutamab is administered once every two (2) weeks. In certain preferred embodiments, the pharmaceutical composition comprising lonigutamab is administered once every three (3) weeks. In certain preferred embodiments, the pharmaceutical composition comprising lonigutamab is administered once every four (4) weeks. In certain embodiments, the pharmaceutical composition comprising lonigutamab is administered once every five (5) weeks. In certain embodiments, the pharmaceutical composition comprising lonigutamab is administered once every six (6) weeks.

In certain preferred embodiments, the pharmaceutical composition comprising lonigutamab is administered at day 1 and day 14. In other preferred embodiments, the pharmaceutical composition comprising lonigutamab is administered at day 1 and day 21. In other preferred embodiments, the pharmaceutical composition comprising lonigutamab is administered at day 1 and day 28.

In certain preferred embodiments, the pharmaceutical composition comprising lonigutamab is subcutaneously administered at day 1 and day 14. In other preferred embodiments, the pharmaceutical composition comprising lonigutamab is subcutaneously administered at day 1 and day 21. In other preferred embodiments, the pharmaceutical composition comprising lonigutamab is subcutaneously administered at day 1 and day 28

### DESCRIPTION OF THE FIGURES

FIG. 1 shows response surfaces for the multimer rate after stress (histidine).

FIG. 2 shows response surfaces to increase the acid variant rate after stress (histidine).

FIG. 3 shows response surfaces for Tₘ₁ (histidine).

FIG. 4 shows response surfaces for Tₘ₂ (histidine)

FIG. 5 shows response surfaces for the multimer rate after stress (phosphate).

FIG. 6 shows response surfaces to increase the acid variant rate after stress (phosphate).

FIG. 7 shows thermal stability: −20° C. DLS results.

FIG. 8 shows thermal stability: 2-8° C. DLS results.

FIG. 9 shows the antibody drug product process flow diagram.

FIG. 10 shows TFF process flow diagram.

FIG. 11 shows representative diagram depicting the infusion bag and ports.

FIG. 12 shows trends for monomer content in in-use compatibility study at room temperature.

FIG. 13 shows trends for main form content in in-use compatibility study at room temperature.

FIG. 14 shows trends for monomer content in in-use compatibility study at 2-8° C.

FIG. 15 shows trends for main form content in in-use compatibility study at 2-8° C.

FIG. 16 shows data pertaining to adverse events after administration of pharmaceutical compositions comprising lonigutamab.

FIG. 17 shows serum concentrations of lonigutamab after intravenous infusion of 0.1, 0.3, 1.0, and 3.0 mg/kg lonigutamab.

FIG. 18 shows serum concentrations of lonigutamab after subcutaneous administration of 20 mg, 40 mg, 125 mg, and 250 mg lonigutamab.

FIG. 19 shows receptor occupancy-time profiles following intravenous infusion of 0.1, 0.3, 1.0, and 3.0 mg/kg lonigutamab.

FIG. 20 shows receptor occupancy-time profiles following subcutaneous administration of 20 mg, 40 mg, 125 mg, and 250 mg lonigutamab.

### DETAILED DESCRIPTION

### General

Provided herein are novel pharmaceutical compositions comprising an antibody, in particular a monoclonal antibody, capable of binding to IGF-1R, as well as methods of making and using such pharmaceutical compositions. From one aspect, provided herein is a novel pharmaceutical composition comprising an antibody, or an antigen binding fragment thereof, capable of binding to IGF-1R and, by inducing internalization of IGF-1R, being internalized into the cell. Also provided herein are certain anti-IGF-1R antibodies comprising a positively charged amino acid at its c-terminus (e.g., a c-terminal arginine, histidine, or lysine). Also provided herein is the use of said pharmaceutical composition and/or antibody to prevent, reduce risk of developing, or treat a disease associated with IGF-1R, such as thyroid eye disease.

Certain aspects of the present disclosure are directed to pharmaceutical compositions and methods of preventing, reducing risk of developing, or treating thyroid eye disease. Thyroid eye disease is a condition in which the eye muscles, eyelids, tear glands and fatty tissues behind the eye become inflamed. This can cause the eyes and eyelids to become red, swollen and uncomfortable and the eyes can be pushed forward (‘staring’ or ‘bulging’ eyes). In some cases there is swelling and stiffness of the muscles that move the eyes so that they no longer move in line with each other; this can cause double vision. Rarely TED can cause reduced vision from pressure on the nerve at the back of the eye or ulcers forming on the front of the eyes if the eyelids cannot close completely.

TED—also known as Graves' Orbitopathy or Ophthalmopathy-is an autoimmune condition. It occurs when the body's immune system attacks the tissue surrounding the eye causing inflammation in the tissues around and behind the eye. In most patients, the same autoimmune condition that causes TED also affects the thyroid gland, resulting in Graves' disease. Graves' disease most commonly causes thyroid overactivity (hyperthyroidism) but can also rarely cause thyroid underactivity (hypothyroidism). TED can occur in people when their thyroid is overactive, underactive or functioning normally. It can also occur after treatment for Graves' disease. People with TED need to be looked after by an eye specialist (ophthalmologist) and a thyroid specialist (endocrinologist).

The currently available therapies for treatment of TED have side-effects, including hearing impairment. These side-effects, which are reported in a substantial proportion of patients receiving currently available therapy, has limited viable therapies of treatment of patients with TED. Thus, there is an unmet need for development of therapies for treatment of TED with greater depth and durability of response.

### Definitions

As used herein the specification, “a” or “an” may mean one or more. As used herein in the claim(s), when used in conjunction with the word “comprising”, the words “a” or “an” may mean one or more than one. For example, reference to an “antibody” is a reference from one to many antibodies. As used herein “another” may mean at least a second or more.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, antibody fragments so long as they exhibit biological activity, and antibody derivatives. The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6. The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The y and a classes are further divided into subclasses (isotypes) on the basis of relatively minor differences in the CH sequence and function, e.g., humans express the following subclasses: IgG1, IgG2, IgG3, IgG4, IgAQ1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

As used herein, the term “CDR” or “complementarity determining region” is intended to mean the non-contiguous antigen binding sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. As used herein, the terms “CDR-L1”, “CDR-L2”, and “CDR-L3” refer, respectively, to the first, second, and third CDRs in a light chain variable region As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

A number of HVR and CDR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

As used in the present specification, the expression “IGF-1R antibody” should be interpreted as similar to “anti-IGF-1R antibody” and means an antibody capable of binding to IGF-1R. In an embodiment of the present application, the epitope of the antibody is localized into the extracellular domain of the human IGF-1R (also referred as IGF-IR ECD). In a particular embodiment, the antibody, or any antigen binding fragment thereof, is capable of binding to IGF-IR with an EC₅₀ comprised between 10×10⁻¹⁰ to 1×10⁻¹⁰, and more preferentially between 8×10⁻¹⁰ to 2×10⁻¹⁰M.

The term “monoclonal antibody” as used herein refers to an antibody obtained from a population of substantially homogeneous antibodies, i.e., the individual antibodies of the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen.

An “antibody fragment” or “antigen-binding fragment” or “functional fragments” of antibodies comprises a portion of an intact antibody, preferably the antigen binding and/or the variable region of the intact antibody or the F region of an antibody which retains or has modified FcR binding capability. Examples of antibody fragments include Fab, Fab′, F(ab′)₂ and Fv fragments; diabodies; and linear antibodies (see U.S. Pat. No. 5,641,870, Example 2; Zapata et al., Protein Eng. 8 (10): 1057-1062 (1995)). Additional examples of antibody fragments include antibody derivatives such as single-chain antibody molecules, monovalent antibodies and multispecific antibodies formed from antibody fragments

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human lgG1, lgG2, lgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

As used herein, a “chimeric antibody” refers to an antibody (immunoglobulin) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or fewer, 9 or fewer, 8 or fewer, 7 or fewer, 6 or fewer, 5 or fewer, 4 or fewer, 3 or fewer, or 2 or fewer. Where pre-existing amino acid changes are present in a VH, preferable those changes occur at only three, two, or one of positions 71H, 73H and 78H; for instance, the amino acid residues at those positions may by 71A, 73T and/or 78A. In some embodiments, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated. (See e.g., Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991)

As used herein, an “interaction” between IGF-1R and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared. The term “preventing” is art-recognized, and when used in relation to a condition, such as thyroid eye disease (TED) related symptoms, relative to a patient who does not receive the therapy.

“Restoring” refers to the act of returning to a normal or healthy condition. The restoration may be partial (e.g., when the subject returns to a condition which is below the normal or healthy condition) or total (e.g., when the subject returns to a condition which is identical or almost identical to a normal or healthy condition). An example of a normal or healthy condition is the visual acuity of a patient prior to Thyroid Eye Disease (TED).

As use herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10⁸ M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment. “Improving vision” refers to the act of enhancing the faculty or state of being able to see, relative to before treatment, including improving acuity, sensitivity, and/or range of visual field.

### Antibodies

In certain aspects, provided herein are pharmaceutical compositions comprising anti-IGF-1R antibodies. Also provided herein are anti-IGF-1R antibodies that have a heavy chain comprising charged amino acid on its c-terminus.

The insulin-like growth factor 1 receptor called IGF-1R (also called IGF1R or IGF-IR) is a receptor with tyrosine kinase activity having 70% homology with the insulin receptor IR. IGF-1R is a glycoprotein of molecular weight approximately 320 kDa. It is a hetero-tetrameric receptor of which each half-linked by disulfide bridges—is composed of an extracellular α-subunit and of a transmembrane β-subunit. IGF-1R binds IGF1 and IGF2 with a very high affinity (Kd #1 nM) but is equally capable of binding to insulin with an affinity 100 to 1000 times lower. Conversely, the IR binds insulin with a very high affinity although the IGFs only bind to the insulin receptor with a 100 times lower affinity. The tyrosine kinase domains of IGF-1R and of IR have a very high sequence homology although the zones of weaker homology respectively concern the cysteine-rich region situated on the α-subunit and the C-terminal part of the β-subunit. The sequence differences observed in the α-subunit are situated in the binding zone of the ligands and are therefore at the origin of the relative affinities of IGF-1R and of IR for the IGFs and insulin respectively. The differences in the C-terminal part of the β-subunit result in a divergence in the signaling pathways of the two receptors; IGF-1R mediating mitogenic, differentiation and anti-apoptosis effects, while the activation of the IR principally involves effects at the level of the metabolic pathways.

The cytoplasmic tyrosine kinase proteins are activated by the binding of the ligand to the extracellular domain of the receptor. The activation of the kinases in its turn involves the stimulation of different intra-cellular substrates, including IRS-1, IRS-2, Shc and Grb 10. The two major substrates of IGF-1R are IRS and She which mediate, by the activation of numerous effectors downstream, the majority of the growth and differentiation effects connected with the attachment of the IGFs to this receptor. The availability of substrates can consequently dictate the final biological effect connected with the activation of the IGF-1R. When IRS-1 predominates, the cells tend to proliferate and to transform. When Shc dominates, the cells tend to differentiate. It seems that the route principally involved for the effects of protection against apoptosis is the phosphatidyl-inositol 3-kinases (PI 3-kinases) route.

In certain embodiments, such antibodies present a high ability to be internalized following IGF-IR binding. As used herein, an antibody that “is internalized” or that “internalized” is one that is taken up by (meaning it “enters”) the cell upon binding to IGF-1R on a mammalian cell. Certain anti-IGF-1R antibodies provided herein are disclosed in U.S. Pat. No. 10,202,458, which is hereby incorporated by reference for the antibodies, antibody sequences, and related compositions that it discloses.

In certain embodiments, the anti-IGF-1R antibody is lonigutamab or an anti-IGF-1R antibody derived from lonigutamab (e.g., sharing at least one CDR, such as CDRH3, with lonigutamab).

In certain embodiments, the anti-IGF-1R antibody comprises a CDRH1 of SEQ ID NO: 1, a CDRH2 of SEQ ID NO: 2, a CDRH3 of SEQ ID NO: 3, a CDRL1 of SEQ ID NO: 4, a CDRL2 of SEQ ID NO: 5, and a CDRL3 of SEQ ID NO: 6, as set forth in the table below.

| CDR | SEQUENCE | SEQ ID NO |
|---|---|---|
| CDRH1 | GYTFTSYY | 1 |
| CDRH2 | IWPGDGST | 2 |
| CDRH3 | ASPMITPNYAMDY | 3 |
| CDRL1 | QDISKY | 4 |
| CDRL2 | YTS | 5 |
| CDRL3 | QQGSTLPYT | 6 |

In some embodiments, the anti-IGF-IR antibody comprises a heavy chain variable region comprising an amino acid sequence of: QVQLVQSGAEVKKPGASVKVSCKASGYTFTSYYIHWVRQAPGQGLEWMGWIWPG DGSTKYAQKFQGRVTMTRDTSTSTVYMELSSLRSEDTAVYFCASPMITPNYAMDY WGQGTLVTVSS (SEQ ID NO:7). In some embodiments, the heavy chain variable region comprises as sequence that is at least 80%, 85%, 90%, 91%, 92%, 93%, 94%, 95%, 96%, 97%, 98%, or 99% identical to SEQ ID NO: 7. In certain embodiments, the heavy chain variable region comprises a sequence that is identical to SEQ ID NO: 7, but for no more than 10, 9, 8, 7, 6, 5, 4, 3, 2, or 1 amino acid substitutions, additions and/or deletions. In certain embodiments, the amino acid substitutions are conservative amino acid substitutions. In some embodiments, the amino acid substitutions, additions and/or deletions occur outside of the heavy chain CDR domains.

In some embodiments, the anti-IGF-1R antibody comprises a light chain variable region comprising an amino acid sequence of: DIQMTQSPSSLSASVGDRVTITCRASQDISKYLNWYQQKPGKAPKLLIYYTSRLQSG VPSRFSGRGSGTDYSLTISSLQPEDFATYFCQQGSTLPYTFGGGTKVEIK (SEQ ID NO: 8). In some embodiments, the light chain variable region comprises as sequence that is at least 80%, 85%, 90%, 91%, 92%, 93%, 94%, 95%, 96%, 97%, 98%, or 99% identical to SEQ ID NO: 8. In certain embodiments, the light chain variable region comprises a sequence that is identical to SEQ ID NO: 8, but for no more than 10, 9, 8, 7, 6, 5, 4, 3, 2, or 1 amino acid substitutions, additions and/or deletions. In certain embodiments, the amino acid substitutions are conservative amino acid substitutions. In some embodiments, the amino acid substitutions, additions and/or deletions occur outside of the light chain CDR domains.

In some embodiments, the anti-IGF-1R antibody comprises a heavy chain comprising an amino acid sequence of: QVQLVQSGAEVKKPGASVKVSCKASGYTFTSYYIHWVRQAPGQGLEWMGWIWPG DGSTKYAQKFQGRVTMTRDTSTSTVYMELSSLRSEDTAVYFCASPMITPNYAMDY WGQGTLVTVSSASTKGPSVFPLAPSSKSTSGGTAALGCLVKDYFPEPVTVSWNSGAL TSGVHTFPAVLQSSGLYSLSSVVTVPSSSLGTQTYICNVNHKPSNTKVDKRVEPKSCD KTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIE KTISKAKGQPREPQVYTLPPSREEMTKNQVSLTCLVKGFYPSDIAVEWESNGQPENN YKTTPPVLDSDGSFFLYSKLTVDKSRWQQGNVFSCSVMHEALHNHYTQKSLSLSPG (SEQ ID NO:9). In some embodiments, the heavy chain comprises as sequence that is at least 80%, 85%, 90%, 91%, 92%, 93%, 94%, 95%, 96%, 97%, 98%, or 99% identical to SEQ ID NO: 9. In certain embodiments, the heavy chain comprises a sequence that is identical to SEQ ID NO: 9, but for no more than 10, 9, 8, 7, 6, 5, 4, 3, 2, or 1 amino acid substitutions, additions and/or deletions. In certain embodiments, the amino acid substitutions are conservative amino acid substitutions. In some embodiments, the amino acid substitutions, additions and/or deletions occur outside of the heavy chain CDR domains.

In some embodiments, the antibody comprises a heavy chain further comprising a charged amino acid at its c-terminus (e.g., a c-terminal arginine, histidine, lysine, aspartic acid, or glutamic acid). In some embodiments, the charged amino acid is a positively charged amino acid (e.g., arginine, histidine, or lysine). In some embodiments the charged amino acid is a negatively charged amino acid (e.g., aspartic acid, or glutamic acid).

In some embodiments, the anti-IGF-1R antibody comprises a heavy chain comprising an amino acid sequence of:

(SEQ ID NO: 11)
QVQLVQSGAEVKKPGASVKVSCKASGYTFTSYYIHWVRQAPGQGLEWMGWIWPG
DGSTKYAQKFQGRVTMTRDTSTSTVYMELSSLRSEDTAVYFCASPMITPNYAMDY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
K.

In some embodiments, the anti-IGF-1R antibody comprises a heavy chain comprising an amino acid sequence of:

(SEQ ID NO: 12)
QVQLVQSGAEVKKPGASVKVSCKASGYTFTSYYIHWVRQAPGQGLEWMGWIWPG
DGSTKYAQKFQGRVTMTRDTSTSTVYMELSSLRSEDTAVYFCASPMITPNYAMDY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
H.

In some embodiments, the anti-IGF-1R antibody comprises a heavy chain comprising an amino acid sequence of:

(SEQ ID NO: 13)
QVQLVQSGAEVKKPGASVKVSCKASGYTFTSYYIHWVRQAPGQGLEWMGWIWPG
DGSTKYAQKFQGRVTMTRDTSTSTVYMELSSLRSEDTAVYFCASPMITPNYAMDY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
R.

In some embodiments, the anti-IGF-IR antibody comprises a heavy chain comprising an amino acid sequence of:

(SEQ ID NO: 14)
QVQLVQSGAEVKKPGASVKVSCKASGYTFTSYYIHWVRQAPGQGLEWMGWIWPG
DGSTKYAQKFQGRVTMTRDTSTSTVYMELSSLRSEDTAVYFCASPMITPNYAMDY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
D.

In some embodiments, the anti-IGF-IR antibody comprises a heavy chain comprising an amino acid sequence of:

(SEQ ID NO: 15)
QVQLVQSGAEVKKPGASVKVSCKASGYTFTSYYIHWVRQAPGQGLEWMGWIWPG
DGSTKYAQKFQGRVTMTRDTSTSTVYMELSSLRSEDTAVYFCASPMITPNYAMDY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
E.

In some embodiments, the anti-IGF-1R antibody comprises a light chain comprising an amino acid sequence of: DIQMTQSPSSLSASVGDRVTITCRASQDISKYLNWYQQKPGKAPKLLIYYTSRLQSG VPSRFSGRGSGTDYSLTISSLQPEDFATYFCQQGSTLPYTFGGGTKVEIKRTVAAPSV FIFPPSDEQLKSGTASVVCLLNNFYPREAKVQWKVDNALQSGNSQESVTEQDSKDST YSLSSTLTLSKADYEKHKVYACEVTHQGLSSPVTKSFNRGEC (SEQ ID NO: 10). In some embodiments, the light chain comprises as sequence that is at least 80%, 85%, 90%, 91%, 92%, 93%, 94%, 95%, 96%, 97%, 98%, or 99% identical to SEQ ID NO: 10. In certain embodiments, the light chain comprises a sequence that is identical to SEQ ID NO: 10, but for no more than 10, 9, 8, 7, 6, 5, 4, 3, 2, or 1 amino acid substitutions, additions and/or deletions. In certain embodiments, the amino acid substitutions are conservative amino acid substitutions. In some embodiments, the amino acid substitutions, additions and/or deletions occur outside of the heavy chain CDR domains.

Full-length antibodies may be prepared by the use of recombinant DNA engineering techniques. Such engineered versions include those created, for example, from natural antibody variable regions by insertions, deletions or changes in or to the amino acid sequences of the natural antibodies. Particular examples of this type include those engineered variable region domains containing at least one CDR and optionally one or more framework amino acids from one antibody and the remainder of the variable region domain from a second antibody. The DNA encoding the antibody may be prepared by deleting all but the desired portion of the DNA that encodes the full length antibody. DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the complementarity determining regions (CDRs) derived substantially or exclusively from the corresponding human antibody regions and DNA encoding CDRs derived substantially or exclusively from a mammal other than a human.

Suitable sources of DNA molecules that encode antibodies include cells, such as hybridomas, that express the full length antibody. For example, the antibody may be isolated from a host cell that expresses an expression vector that encodes the heavy and/or light chain of the antibody.

Antibody fragments, including but not limited to Fab fragments, and/or antibody derivatives may also be prepared by the use of recombinant DNA engineering techniques involving the manipulation and re-expression of DNA encoding antibody variable and constant regions. Standard molecular biology techniques may be used to modify, add or delete further amino acids or domains as desired. Any alterations to the variable or constant regions are still encompassed by the terms ‘variable’ and ‘constant’ regions as used herein. In some instances, PCR is used to generate an antibody fragment by introducing a stop codon immediately following the codon encoding the interchain cysteine of C_{H}1, such that translation of the C_{H}1 domain stops at the interchain cysteine. Methods for designing suitable PCR primers are well known in the art and the sequences of antibody C_{H}1 domains are readily available. In some embodiments, stop codons may be introduced using site-directed mutagenesis techniques.

An antibody of the present disclosure may be derived from any antibody isotype (“class”) including for example IgG, IgM, IgA, IgD and IgE and subclasses thereof, including for example IgG1, IgG2, IgG3 and IgG4. In certain preferred embodiments, the heavy and light chains of the antibody are from IgG. The heavy and/or light chains of the antibody may be from murine IgG or human IgG. In certain other preferred embodiments, the heavy and/or light chains of the antibody are from human IgG1. In still other preferred embodiments, the heavy and/or light chains of the antibody are from human IgG4.

An antibody of the present disclosure may be a monoclonal antibody, a polyclonal antibody, a recombinant antibody, a humanized antibody, a human antibody, a chimeric antibody, a multispecific antibody, an antibody fragment thereof, or a derivative thereof. In some embodiments, the antibody is humanized antibody.

The antibodies of the present disclosure may also be an antibody fragment, such as a Fab fragment, a Fab′ fragment, a F(ab′)₂ fragment, a Fv fragment, a diabody, or a single chain antibody molecule. In some embodiments, the antibody fragment is a Fab fragment.

In some embodiments, antibodies are human monoclonal antibodies which may be prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline and/or non-germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

In some embodiments, antibodies are humanized and/or chimeric monoclonal antibodies, which can be raised by immunizing rodents (e.g., mice, rats, hamsters and guinea pigs) with either (1) the native IGF-IR derived from enzymatic digestion of a purified IGF-IR from human plasma or serum, or (2) a recombinant IGF-1R, or its derived fragment, expressed by either eukaryotic or prokaryotic systems. Other animals can be used for immunization, e.g., non-human primates, transgenic mice expressing human immunoglobulins, and severe combined immunodeficient (SCID) mice transplanted with human B-lymphocytes.

Polyclonal and monoclonal antibodies are naturally generated as immunoglobulin (Ig) molecules in the immune system's response to a pathogen. A dominating format with a concentration of 8 mg/ml in human serum, the ˜150-kDa IgG1 molecule is composed of two identical ˜50-kDa heavy chains and two identical ˜25-kDa light chains.

Hybridomas can be generated by conventional procedures by fusing B-lymphocytes from the immunized animals with myeloma cells. In addition, antibodies can be generated by screening recombinant single-chain Fv or Fab libraries from human B-lymphocytes in a phage-display system. The specificity of the MAbs to human IGF-IR can be tested by enzyme linked immunosorbent assay (ELISA), Western immunoblotting, or other immunochemical techniques.

### Nucleic Acids, Vectors and Host Cells

Antibodies suitable for use in the methods of the present disclosure may be produced using recombinant methods and compositions, e.g., as described in U.S. Pat. No. 4,816,567. In some embodiments, isolated nucleic acids having a nucleotide sequence encoding any of the antibodies of the present disclosure are provided. Such nucleic acids may encode an amino acid sequence containing the V_{L}/C_{L} and/or an amino acid sequence containing the V_{H}/C_{H}1 of the anti-IGF-IR antibody. In some embodiments, one or more vectors (e.g., expression vectors) containing such nucleic acids are provided. A host cell containing such nucleic acid may also be provided. The host cell may contain (e.g., has been transduced with): (1) a vector containing a nucleic acid that encodes an amino acid sequence containing the V_{L}/C_{L} of the antibody and an amino acid sequence containing the V_{H}/C_{H}1 of the antibody, or (2) a first vector containing a nucleic acid that encodes an amino acid sequence containing the V_{L}/C_{L} of the antibody and a second vector containing a nucleic acid that encodes an amino acid sequence containing the V_{H}/C_{H}1 of the antibody. In some embodiments, the host cell is eukaryotic, e.g., a Chinese Hamster Ovary (CHO) cell or lymphoid cell (e.g., Y0, NS0, Sp20 cell). In some embodiments, the host cell is a bacterium such as E. coli.

Methods of making an anti-IGF-IR antibody are disclosed herein. The method includes culturing a host cell of the present disclosure containing a nucleic acid encoding the anti-IGF-1R antibody, under conditions suitable for expression of the antibody. In some embodiments, the antibody is subsequently recovered from the host cell (or host cell culture medium).

For recombinant production of a humanized anti-IGF-1R antibody of the present disclosure, a nucleic acid encoding the antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Stratagene, and Invitrogen.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-IGF-1R antibody of the present disclosure.

Suitable host cells for cloning or expression of antibody-encoding vectors include prokaryotic or eukaryotic cells. For example, an anti-IGF-IR antibody of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, NJ, 2003), pp. 245-254, describing expression of antibody fragments in E. coli.). In other embodiments, the antibody of the present disclosure may be produced in eukaryotic cells, e.g., a Chinese Hamster Ovary (CHO) cell or lymphoid cell (e.g., Y0, NS0, Sp20 cell) (e.g., U.S. patent application Ser. No. 14/269,950, U.S. Pat. No. 8,981,071, Eur J Biochem. 1991 Jan. 1; 195 (1): 235-42). After expression, the antibody may be isolated from the bacterial cell paste in a soluble fraction and can be further purified.

### Pharmaceutical Compositions and Administration

The present disclosure is generally directed to pharmaceutical compositions comprising anti-IGF-1R antibodies disclosed herein. As used herein, the expression “pharmaceutical composition (also referred to as a “pharmaceutical formulation”) means a combination of at least one active ingredient (e.g., an anti-IGF-1R antibody disclosed herein), and at least one inactive ingredient which, when combined with the active ingredient and/or one or more additional inactive ingredients, is suitable for therapeutic administration to a human or non-human animal.

In certain aspects, provided herein is a pharmaceutical composition comprising: (a) at least 75 mg/ml of an anti-IGF-1R antibody; (b) from 20 to 30 mM histidine; and (c) from 4% to 6% D-sorbitol; wherein the pharmaceutical composition is at a pH of from 5.5-6.5.

In certain embodiments, the pharmaceutical composition comprises at least 75 mg/ml of anti-IGF-1R antibody. In some embodiments, the pharmaceutical composition comprises at least 100 mg/ml, at least 125 mg/ml, at least 150 mg/ml, at least 175 mg/ml, at least 200 mg/ml, or at least 250 mg/ml of the anti-IGF-IR antibody. In certain embodiments, the pharmaceutical composition comprises from 75 mg/ml to 300 mg/ml, from 100 mg/ml to 300 mg/ml, or from 125 mg/ml to 250 mg/ml of the anti-IGF-IR antibody. In some embodiments, the pharmaceutical composition comprises about 125 mg/ml, about 150 mg/ml, about 175 mg/ml, about 200 mg/ml, or about 250 mg/ml of the anti-IGF-1R antibody.

In some embodiments, the pharmaceutical composition comprises from 20 to 30 mM histidine. In certain embodiments, the pharmaceutical composition comprises about 20 mM, about 21 mM, about 22 mM, about 23 mM, about 24 mM, about 25 mM, about 26 mM, about 27 mM, about 28 mM, about 29 mM, or about 30 mM histidine.

In some embodiments, the pharmaceutical composition comprises from 4% to 6% D-sorbitol. In certain embodiments, the pharmaceutical composition comprises about 4%, 5%, or 6% D-sorbitol.

In some embodiments, the pharmaceutical composition is at a pH of from 5.5-6.5. In certain embodiments, the pharmaceutical composition is at a pH of about 5.5, about 5.6, about 5.7, about 5.8, about 5.9, about 6.0, about 6.1, about 6.2, about 6.3, about 6.4, or about 6.5.

In some embodiments, the pharmaceutical composition does not comprise polysorbate 80. In some embodiments, the pharmaceutical composition comprises a small amount of polysorbate 80 (e.g., less than 0.05%). In certain embodiments, the pharmaceutical composition comprises no more than 0.05% polysorbate 80. In some embodiments, the pharmaceutical composition comprises no more than 0.02% polysorbate 80. In some embodiments, the pharmaceutical composition comprises no more than 0.01% polysorbate 80. In some embodiments, the pharmaceutical composition comprises no more than 0.005% polysorbate 80. In certain embodiments, the pharmaceutical composition further comprises from 0.002% to 0.05% polysorbate 80. In some embodiments, the pharmaceutical composition comprises about 0.002%, about 0.003%, about 0.004%, about 0.005%, about 0.006%, about 0.007%, about 0.008%, about 0.009%, about 0.01%, about 0.015%, about 0.02%, about 0.025%, about 0.03%, about 0.035%, about 0.04%, about 0.045%, or about 0.05% polysorbate 80.

In some embodiments, the pharmaceutical composition does not comprise poloxamer 188. In some embodiments, the pharmaceutical composition comprises a small amount of poloxamer 188 (e.g., less than 0.1%) as a surfactant. In certain embodiments, the pharmaceutical composition comprises no more than 0.1% poloxamer 188. In some embodiments, the pharmaceutical composition comprises no more than 0.05% poloxamer 188. In some embodiments, the pharmaceutical composition comprises no more than 0.02% poloxamer 188. In some embodiments, the pharmaceutical composition comprises no more than 0.01% poloxamer 188. In some embodiments, the pharmaceutical composition further comprises from 0.01% to 0.1% poloxamer 188. In certain embodiments, the pharmaceutical composition comprises about 0.01%, about 0.02%, about 0.03%, about 0.04%, about 0.05%, about 0.06%, about 0.07%, about 0.08%, about 0.09%, or about 0.1% poloxamer 188.

In certain embodiments, the pharmaceutical compositions provided herein exhibit high levels of stability. The term “stable,” as used herein in reference to the pharmaceutical compositions, means that the antibodies within the pharmaceutical compositions retain an acceptable degree of structure and/or function and/or biological activity after storage for a defined amount of time. A composition may be stable even though the antibody contained therein does not maintain 100% of its structure and/or function and/or biological activity after storage for a defined amount of time. Under certain circumstances, maintenance of about 80%, about 85%, about 90%, about 95%, about 96%, about 97%, about 98% or about 99% of an antibody's structure and/or function and/or biological activity after storage for a defined amount of time may be regarded as “stable.”

Stability can be measured, inter alia, by determining the percentage of native antibody remaining in the composition after storage for a defined amount of time at a given temperature. The percentage of native antibody can be determined by, inter alia, size exclusion chromatography (e.g., size exclusion high performance liquid chromatography [SE-HPLC]). An “acceptable degree of stability,” as that phrase is used herein, means that at least 90% of the monomeric form of the antibody can be detected in the composition after storage for a defined amount of time at a given temperature. In certain embodiments, at least about 90%, 91%, 92%, 93%, 94%, 95%, 96%, 97%, 98%, 99% or 100% of the monomeric form of the antibody can be detected in the composition after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical composition may be stored when assessing stability can be any temperature from about −80° C. to about 45° C., e.g., storage at about −30° C., about −20° C., about 0° C., about 5° C., about 25° C., or about 45° C. For example, a pharmaceutical composition may be deemed stable if after 3 months of storage at 5° C., greater than about 90%, 95%, 96% or 97% of monomeric antibody is detected by SE-HPLC. A pharmaceutical composition may also be deemed stable if after 6 months of storage at 5° C., greater than about 90%, 95%, 96% or 97% of monomeric antibody is detected by SE-HPLC. A pharmaceutical composition may also be deemed stable if after 9 months of storage at 5° C., greater than about 90%, 95%, 96% or 97% of monomeric antibody is detected by SE-HPLC. A pharmaceutical composition may also be deemed stable if after 3 months of storage at 25° C., greater than about 90%, 95%, 96% or 97% of monomeric antibody is detected by SE-HPLC. A pharmaceutical composition may also be deemed stable if after 6 months of storage at 25° C., greater than about 90%, 95%, 96% or 97% of monomeric antibody is detected by SE-HPLC. A pharmaceutical composition may also be deemed stable if after 9 months of storage at 25° C., greater than about 90%, 95%, 96% or 97% of monomeric antibody is detected by SE-HPLC.

Other methods may be used to assess the stability of the pharmaceutical compositions disclosed herein, such as, e.g., differential scanning calorimetry (DSC) to determine thermal stability, controlled agitation to determine mechanical stability, and absorbance at about 350 nm or about 405 nm to determine solution turbidities. For example, a pharmaceutical composition disclosed herein may be considered stable if, after 6 or more months of storage at about 5° C. to about 25° C., the change in OD₄₀₅ of the composition is less than about 0.05 (e.g., 0.04, 0.03, 0.02, 0.01, or less) from the OD₄₀₅ of the composition at t=0.

Stability may also be assessed by measuring the biological activity and/or binding affinity of the antibody to its target. For example, a composition disclosed herein may be regarded as stable if, after storage at e.g., 5° C., 25° C., 45° C., etc. for a defined amount of time (e.g., 1 to 12 months), the anti-IGF-1R antibody contained within the composition binds to IGF-1R with an affinity that is at least 50%, 60%, 70%, 80%, 90%, 95%, or more of the binding affinity of the antibody prior to said storage. Additional methods for assessing the stability of an antibody in composition are demonstrated in the Examples presented below.

In certain embodiments, after 12 weeks at −20° C. and 2-8° C., the pharmaceutical compositions provided herein continue to display very good stability in terms of low aggregation; confirmed by both visual assessment and SEC-HPLC. The data from SEC-HPLC reveals no appreciable increase in high molecular weight species (HMWS) from T0-T12w, with both temperatures performing comparably. CEX-HPLC analysis reveals that the compositions show good chemical stability after 12 weeks at −20° C. and 2-8° C. From the CEX-HPLC data, slightly higher values for % main species are observed for samples kept at 2-8° C. This indicates that the freeze-thaw cycles undergone by samples at −20° C. do not affect aggregation but are detrimental to the chemical stability of the molecule. Improvement to chemical stability is gained from formulation 3a (in Example 1, seen at both 2-8° C. and −20° C.) which contains sorbitol as tonicity modifier with no added PS80.

DLS measurements reveal that particle size ZD is virtually unchanged after 12 weeks at both temperatures. PDI values show a trend towards increasing values over the timepoints, but all compositions remain monodisperse after 12 weeks.

In some embodiments, the pharmaceutical composition is stable for at least 8 weeks, at least 9 weeks, at least 10 weeks, at least 11 weeks, at least 12 weeks, at least 13 weeks, at least 14 weeks, at least 15 weeks, or at least 16 weeks. In some embodiments, the pharmaceutical composition is stable at a temperature of from −20° C. to 8° C.

In the fluid form, the pharmaceutical compositions provided herein may, in certain embodiments, exhibit low to moderate levels of viscosity. “Viscosity” as used herein may be “kinematic viscosity” or “absolute viscosity.” “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. For example, if one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density (Absolute Viscosity=Kinematic Viscosity×Density). The dimension of kinematic viscosity is L²/T where L is a length and T is a time. Commonly, kinematic viscosity is expressed in centistokes (cSt). The System International (SI) unit of kinematic viscosity is mm²/s, which is 1 cSt. Absolute viscosity is expressed in units of centipoise (cP). The SI unit of absolute viscosity is the milliPascal-second (mPa·s), where 1 cP=1 mPa·s.

As used herein, a low level of viscosity, in reference to a pharmaceutical composition disclosed herein, will exhibit an absolute viscosity of less than about 20 cPoise (cP). For example, a pharmaceutical composition disclosed herein will be deemed to have “low viscosity,” if, when measured using standard viscosity measurement techniques, the composition exhibits an absolute viscosity of about 19 cP, about 18 cP, about 17 cP, about 16 cP, about 15 cP, about 14 cP, about 13 cP, about 12 cP, about 11 cP, about 10 cP, about 9 cP, about 8 cP, about 7 cP, about 6 cP, about 5 cP, about 4 cP, or less at 21° C. As used herein, a moderate level of viscosity, in reference to a pharmaceutical composition disclosed herein, will exhibit an absolute viscosity of between about 30 cP and about 20 cP at 21° C. For example, a pharmaceutical composition disclosed herein will be deemed to have “moderate viscosity,” if when measured using standard viscosity measurement techniques, the composition exhibits an absolute viscosity of about 30 cP, about 29 cP, about 28 cP, about 27 cP, about 26 cP, about 25 cP, about 24 cP, about 23 cP, about 22 cP, about 21 cP or about 20 cP at 21° C.

In some embodiments, the osmolality of the pharmaceutical composition is within physiological osmolality range of 250-400 mOsm/kg. In some embodiments, the viscosity of the pharmaceutical composition is no more than 30 cP at 21° C. In some embodiments, the viscosity of the pharmaceutical composition is no more than 15 cP at 21° C. In some embodiments, the viscosity of the pharmaceutical composition is about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, about 23 cP, about 24 cP, about 25 cP, about 26 cP, about 27 cP, about 28 cP, about 29 cP, or about 30 cP at 21° C.

### Injectors

In certain aspects, the present disclosure relates to an injector comprising the pharmaceutical composition disclosed herein. In some embodiments, the injector comprises a delivery volume of no more than 2 ml. In some embodiments, the injector comprises a needle of a size of no bigger than 24G (e.g., 25G, 27G) . . . . In some embodiments, the injector is an automatic reusable fix dose Pen. In some embodiments, the injector is an automatic reusable variable dose Pen. In some embodiments, the injector is an automatic disposable fix dose autoinjector.

In some embodiments, the pharmaceutical compositions provided herein may be contained within any container suitable for storage of medicines and other therapeutic compositions. For example, the pharmaceutical compositions may be contained within a sealed and sterilized plastic or glass container having a defined volume such as a vial, ampule, syringe, cartridge, or bottle. Different types of vials can be used to contain the compositions provided herein, including, e.g., clear and opaque (e.g., amber) glass or plastic vials. Likewise, any type of syringe can be used to contain and/or administer the pharmaceutical compositions disclosed herein.

The pharmaceutical compositions provided herein may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The pharmaceutical compositions provided herein may be contained within plastic syringes. In the last decade, pharmaceutical protein and peptide drug products have been approved for use with prefilled plastic syringes. For example, the pharmaceutical compositions may be contained in a Daikyo Crystal Zenith (CZ) syringe (Daikyo Seiko, Tokyo).

The rubber plungers used in syringes, and the rubber stoppers used to close the openings of vials, may be coated to prevent contamination of the medicinal contents of the syringe or vial and/or to preserve their stability. Thus, pharmaceutical compositions provided herein, according to certain embodiments, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical compositions disclosed herein are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the methods disclosed herein are commercially available under the tradename “FluroTec®,” available from West Pharmaceutical Services, Inc. (Lionville, Pa.).

In certain embodiments, the pharmaceutical compositions can be administered to a patient by parenteral routes such as injection (e.g., subcutaneous, intravenous, intramuscular, intraperitoneal, infraorbital, intravitreal, intraocular, subconjunctival, retrobulbar, peribulbar, and/or intrathecal injection). Numerous reusable pen and/or autoinjector delivery devices can be used to subcutaneously deliver the pharmaceutical compositions disclosed herein. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition disclosed herein include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park, Ill.), to name only a few.

The use of West SelfDose and SmartDose injection systems to deliver the pharmaceutical compositions disclosed herein is also contemplated herein. The use of a microinfusor to deliver the pharmaceutical compositions disclosed herein is also contemplated herein. As used herein, the term “microinfusor” means a subcutaneous delivery device designed to slowly administer large volumes (e.g., up to about 2.5 mL or more) of a therapeutic composition over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996).

### Methods of Treatment

In certain aspects, the present disclosure relates to methods of preventing, reducing risk of developing, or treating a disease or disorder associated with IGF-IR comprising administering the pharmaceutical composition and/or an anti-IGF-IR antibody disclosed herein. The present disclosure is also generally directed to methods of preventing, reducing risk of developing, or treating IGF-IR related diseases and disorders comprising administering the anti-IGF-1R antibody disclosed herein. The present disclosure is also generally directed to methods of preventing, reducing risk of developing, or treating IGF-1R related diseases and disorders comprising administering the pharmaceutical composition using the injector disclosed herein. In some embodiments, the disease or disorder is thyroid eye disease (TED).

In some embodiments, the disease or disorder is stroke, acromegaly, diabetic nephropathy (diabetic kidney disease), idiopathic pulmonary fibrosis, interstitial lung disease, obesity, type 2 diabetes, juvenile idiopathic arthritis (JIA), diffuse cutaneous systemic sclerosis, Sjogren's syndrome calcinosis and vasculitis, cachexia and sarcopenia, diabetic macular edema, arterial atherosclerosis, peripheral artery disease (PAD), myocardial infarction and stroke, diabetic foot and skin, rheumatoid arthritis, neurofibromatosis 1, neurofibromatosis 2, polycystic kidney disease, polycystic liver disease, polycystic ovarian syndrome, Alzheimers disease, cognitive decline, dementia, depression and anxiety, asthma, aging, thyroid eye disease, idiopathic orbital inflammation, human type 2 lipodystrophy and associated cardiomyopathy, autosomal dominant polycystic kidney disease (ADPKD), NASH, Graves disease, and/or Hashimoto's thyroiditis.

In some embodiments, the disease or disorder is thyroid eye disease (TED). Thyroid-associated ophthalmopathy (TAO), also known as thyroid eye disease (TED), Graves' ophthalmopathy or orbitopathy (GO), thyrotoxic exophthalmos, dysthyroid ophthalmopathy, and several other terms, is orbitopathy associated with thyroid dysfunction. TED is divided into two types. Active TED, which typically lasts 1-3 years, is characterized by an ongoing autoimmune/inflammatory response in the soft tissues of the orbit. Active TED is responsible for the expansion and remodeling of the ocular soft tissues. The autoimmune/inflammatory response of active TED spontaneously resolves and the condition transitions into inactive TED. Inactive TAO is the term used to describe the long-term/permanent sequelae of active TED. The cause of TED is unknown. TED is typically associated with Graves' hyperthyroidism, but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets—a phenomenon termed proptosis or exophthalmos. Although most cases of TED do not result in loss of vision, this condition can cause vision-threatening exposure keratopathy, troublesome diplopia (double vision), and compressive dysthyroid optic neuropathy. TED may precede, coincide with, or follow the systemic complications of dysthyroidism. The ocular manifestations of TED include upper eyelid retraction, lid lag, swelling, redness (erythema), conjunctivitis, and bulging eyes (exophthalmos or proptosis), chemosis, periorbital edema, and altered ocular motility with significant functional, social, and cosmetic consequences. Many of the signs and symptoms of TED, including proptosis and ocular congestion, result from expansion of the orbital adipose tissue and periocular muscles. The adipose tissue volume increases owing in part to new fat cell development (adipogenesis) within the orbital fat. The accumulation of hydrophilic glycosaminoglycans, primarily hyaluronic acid, within the orbital adipose tissue and the perimysial connective tissue between the extraocular muscle fibers, further expands the fat compartments and enlarges the extraocular muscle bodies. Hyaluronic acid is produced by fibroblasts residing within the orbital fat and extraocular muscles, and its synthesis in vitro is stimulated by several cytokines and growth factors, including IL-1beta, interferon-gamma, platelet-derived growth factor, thyroid stimulating hormone (TSH) and insulin-like growth factor I (IGF-I).

Antibodies that activate the insulin-like growth factor I receptor (IGF-IR) have also been detected and implicated in active TED. Without being bound to any theory, it is believed that TSHR and IGF-IR form a physical and functional complex in orbital fibroblasts, and that blocking IGF-IR appears to attenuate both IGF-1 and TSH-dependent signaling. It has been suggested that blocking IGF-IR using an antibody antagonist might reduce both TSHR- and IGF-I-dependent signaling and therefore interrupt the pathological activities of autoantibodies acting as agonists on either receptor.

IGF-IR is a widely expressed heterotetrameric protein involved in the regulation of proliferation and metabolic function of many cell types. It is a tyrosine kinase receptor comprising two subunits. IGF-IRalpha contains a ligand-binding domain while IGF-IRbeta is involved in signaling and contains tyrosine phosphorylation sites.

Thyroid Eye Disease (TED), is a debilitating autoimmune disorder that occurs in patients with Graves' Disease in which inflammation in the muscle and fat tissue behind the eyes results in proptosis, diplopia, redness, pain, and swelling, leading to photosensitivity, blurred vision, and in serious cases, blindness. The mechanistic underpinnings of TED involve a complex interaction between autoantibody-mediated stimulation of Thyroid Stimulating Hormone Receptor (TSHR) and Insulin-like growth factor 1 receptor (IGF-1R) signaling in orbital fibroblasts that cause orbital tissue inflammation and expansion. Current therapies include corticosteroids and teprotumumab, as well as surgical intervention to prevent vision loss. Lonigutamab is a high-affinity (K_{D}<50 pM) monoclonal antibody directed against IGF-1R that induces rapid and efficient receptor internalization. Lonigutamab is being developed as a potential treatment for TED. To support clinical development of Lonigutamab, a multi-color flow cytometric assay was developed to monitor the binding of Lonigutamab to IGF-IR on the surface of human Peripheral Blood Mononuclear Cells (PBMCs).

Lonigutamab is a humanized monoclonal antibody against IGF-1R. Lonigutamab has high affinity and specificity toward IGF-1R. Specifically, lonigutamab has picomolar affinity for IGF-1R. In certain preferred embodiments, lonigutamab has a ko of about 30 pM to a binding epitope of IGF-1R. Once lonigutamab binds to IGF-1R, it induces receptor internalization, which results in signal blockade from IGF-IR, which may lead to the therapeutic effect of lonigutamab. The IGF-1R internalization may occur within minutes after administration of pharmaceutical compositions comprising lonigutamab. In certain preferred embodiments, the therapeutically effective serum concentration of lonigutamab may be attained in about an hour after administration of the pharmaceutical composition comprising lonigutamab.

Lonigutamab has unique and beneficial pharmacological properties. In certain preferred embodiments, administration of pharmaceutical compositions comprising lonigutamab induce greater than about 70% IGF-IR internalization. In certain preferred embodiments, administration of pharmaceutical compositions comprising lonigutamab induce greater than about 80% IGF-IR internalization. In certain preferred embodiments, administration of pharmaceutical compositions comprising lonigutamab induce greater than about 85% IGF-IR internalization. In certain preferred embodiments, administration of pharmaceutical compositions comprising lonigutamab induce greater than about 90% IGF-IR internalization. In certain preferred embodiments, administration of pharmaceutical compositions comprising lonigutamab induce greater than about 95% IGF-1R internalization. In certain preferred embodiments, administration of pharmaceutical compositions comprising lonigutamab induce greater than about 97% IGF-IR internalization. In certain preferred embodiments, administration of pharmaceutical compositions comprising lonigutamab induce greater than about 99% IGF-1R internalization.

In certain embodiments of the compositions and methods provided herein, the pharmaceutical composition is formulated for subcutaneous administration. In some embodiments, the pharmaceutical composition is administered subcutaneously. In certain embodiments, the pharmaceutical composition is formulated for intramuscular administration. In certain embodiments, the pharmaceutical composition is administered intramuscularly. In some embodiments, the pharmaceutical composition is formulated for infraorbital, intravitreal, intraocular, subconjunctival, retrobulbar, peribulbar, and/or intrathecal administration. In certain embodiments, the pharmaceutical composition is administered by infraorbital, intravitreal, intraocular, subconjunctival, retrobulbar, peribulbar, and/or intrathecal injection.

In certain embodiments, the pharmaceutical composition is administered in a delivery volume of no more than 3 ml, 2.5 ml, 2 ml, 1.5 ml, or 1 ml. In some embodiments, the pharmaceutical composition is administered in a delivery volume of no more than 2 ml.

In certain embodiments, the pharmaceutical composition is administered via a needle of a size of no bigger than 24G, 25G, or 27G. In some embodiments, the pharmaceutical composition is administered in a 24G needle, a 25G needle, or a 27G needle.

In certain embodiments, the pharmaceutical composition is administered with an injection force of no more than 14N, 13N, 12N, 11N, 10N, 9N, 8N, 7N, or 6N. In some embodiments, the pharmaceutical composition is administered with an injection force of no more than 12N. In certain embodiments, the pharmaceutical composition is administered with an injection force of about 4N, about 5N, about 6N, about 7N, about 8N, about 9N, about 10N, about 11N, or about 12N.

In some embodiments, the method reduces the severity of the thyroid eye disease (TED). In some embodiments, the method reduces proptosis in an eye in a subject with thyroid eye disease (TED). In some embodiments, proptosis is reduced by at least 2 mm. In some embodiments, proptosis is reduced by at least 3 mm. In some embodiments, proptosis is reduced by at least 4 mm.

In some embodiments, the method reduces Clinical Activity Score (CAS) of thyroid eye disease (TED). In some embodiments, the clinical activity score (CAS) is reduced by at least 2 points. In some embodiments, the clinical activity score (CAS) is reduced to one (1). In some embodiments, the clinical activity score (CAS) of the subject is reduced to zero (0). In some embodiments, the method improves the quality of life in the subject. In some embodiments, the quality of life is measured by the Graves' Ophthalmopathy Quality of Life (GO-QoL) assessment. In some embodiments, the quality of life is measured by the Visual Functioning or Appearance subscale thereof. In some embodiments, the quality of life is measured by the European Group on Graves' orbitopathy (EUGOGO) guidelines. In some embodiments, the method reduces the severity of diplopia. In some embodiments, the diplopia is constant diplopia. In some embodiments, the diplopia is inconstant diplopia. In some embodiments, the diplopia is intermittent diplopia.

In certain aspects, the invention s methods of administration of pharmaceutical compositions comprising lonigutamab. These pharmaceutical compositions may be administered to patients suffering from thyroid eye disease (TED). In certain embodiments, the pharmaceutical compositions of the invention may be administered intravenously. The methods of the invention provide intravenous infusion of pharmaceutical compositions comprising up to about 3.0 mg/kg lonigutamab to the patient. In certain embodiments, methods of the invention provide administration of pharmaceutical compositions comprising about 0.1, 0.3, 1.0, or 3.0 mg/kg of lonigutamab as an intravenous infusion. The invention further provides that the intravenous infusion may take place for a period of about 15 minutes to about 120 minutes. In certain preferred embodiments, the intravenous infusion takes place for a duration of about 60 minutes.

In certain aspects, the invention provides administration of pharmaceutical compositions comprising lonigutamab as a subcutaneous injection. In certain embodiments, the methods of the invention provide subcutaneous administration of up to about 250 mg lonigutamab. In certain embodiments, the pharmaceutical composition for subcutaneous administration comprises about 20 mg, 40 mg, 125 mg, or 250 mg of lonigutamab. The pharmaceutical compositions for subcutaneous administration may have a volume of from about 0.5 mL to about 3 mL. In preferred embodiments, the administered volume is up to about 2 mL. Beneficially, since the subcutaneous administration is conducted within tolerable volumes, the pharmaceutical compositions could be administered in an out-patient setting, or the pharmaceutical compositions of the invention could be self-administered by the patients. This results in a convenient and an efficient way for administration of the pharmaceutical composition comprising lonigutamab. In particular, the subcutaneous administration of pharmaceutical compositions is beneficial as compared to the intravenous administration due to the ease of administration of the pharmaceutical composition.

In certain embodiments of the invention, the administration of pharmaceutical composition comprising lonigutamab results in maximal IGF-IR receptor occupancy with the anti-IGF-1R antibody. This maximal occupancy of IGF-IR may be achieved any time after the administration of the pharmaceutical composition comprising lonigutamab. In certain embodiments, this is achieved after about 12 hours after administration of the pharmaceutical composition comprising lonigutamab. The pharmaceutical compositions administered may comprise about 100 mg to about 300 mg lonigutamab. In preferred embodiments, the pharmaceutical composition may comprise about 125 mg or about 250 mg of lonigutamab. Notably, the maximal IGF-IR receptor occupancy may be maintained up to a few months after a single administration. In preferred embodiments, the IGF-IR receptor occupancy maintained for at least about 4 weeks after a single administration of a pharmaceutical composition comprising lonigutamab. The maximal receptor occupancy level may be maintained after a single subcutaneous injection or an intravenous infusion of pharmaceutical composition comprising lonigutamab.

Teprotumumab, a previously known antibody for IGF-IR occupancy was maintained a concentration of 200 μg/mL to have improved efficacy. Lonigutamab is approximately 75 times more potent as compared to teprotumumab, the equivalent serum concentration required to maintain therapeutic activity of lonigutamab is predicted to be around 3 μg/mL. In certain aspects, the serum concentration required for maximal IGF-IR receptor internalization is a concentration of approximately about 3 μg/mL of lonigutamab. Thus, the therapeutically effective serum concentration of lonigutamab is at least about 1 μg/mL. In certain preferred embodiments, the therapeutically effective serum concentration of lonigutamab is at least about 3 μg/mL. Importantly, the pharmaceutical compositions of the invention provide a therapeutically effective serum concentration of lonigutamab to the patient.

In certain aspects of the invention, the pharmaceutical compositions comprising lonigutamab administered to the patients are safe and tolerable. Moreover, the pharmaceutical compositions of the invention have minimal adverse events.

In certain aspects, the invention provides methods of treatment of thyroid eye disease (TED) comprising subcutaneous administration of a pharmaceutical composition comprising from about 10 mg to about 500 mg lonigutamab. In certain embodiments, the pharmaceutical composition comprises about 20 mg lonigutamab. In certain embodiments, the pharmaceutical composition comprises about 40 mg lonigutamab. In certain embodiments, the pharmaceutical composition comprises about 125 mg lonigutamab. In certain embodiments, the pharmaceutical composition comprises about 250 mg lonigutamab.

In certain embodiments, the pharmaceutical composition comprising lonigutamab is administered once weekly. In certain embodiments, the pharmaceutical composition comprising lonigutamab is administered twice weekly. In certain embodiments, the pharmaceutical composition comprising lonigutamab is administered thrice weekly. In certain embodiments, the pharmaceutical composition comprising lonigutamab is administered on alternate days, i.e., once every two days. In certain preferred embodiments, the pharmaceutical composition comprising lonigutamab is administered once every two (2) weeks. In certain preferred embodiments, the pharmaceutical composition comprising lonigutamab is administered once every three (3) weeks. In certain preferred embodiments, the pharmaceutical composition comprising lonigutamab is administered once every four (4) weeks. In certain embodiments, the pharmaceutical composition comprising lonigutamab is administered once every five (5) weeks. In certain embodiments, the pharmaceutical composition comprising lonigutamab is administered once every six (6) weeks.

In certain preferred embodiments, the pharmaceutical composition comprising lonigutamab is administered at day 1 and day 14. In other preferred embodiments, the pharmaceutical composition comprising lonigutamab is administered at day 1 and day 21. In other preferred embodiments, the pharmaceutical composition comprising lonigutamab is administered at day 1 and day 28.

### EXAMPLES

### Example 1: Pre-Formulation Development of the Anti Human IGF-IR mAb

The drug product formulation was prepared for the high dose of 125 mg/ml of the anti-IGF-1R antibody, where the concentration of the active pharmaceutical ingredient (API) is higher than that of the drug substance at 20 mg/mL. The composition of excipients remains the same in both 20 mg/mL and 125 mg/mL formulations.

The anti-IGF-1R antibody is formulated in an isotonic, sterile solution manufactured at two concentrations, 20 mg/mL and 125 mg/mL, to enable delivery by IV and SC administration. The composition of the drug product is the same as that of the drug substance in both product formulations. The drug product is presented in a single-dose, depyrogenated Type 1 glass vial.

The Process 1 drug substance was manufactured at 20 mg/mL in the formulation buffer containing 25 mM histidine, 6% sucrose, pH 6.0. There was no process development performed for the manufacturing of the 20 mg/mL batch. The process consists of sterile filtration through two filters installed in series and the fill-finish operations.

For the 125 mg/mL drug product (DP) production, the manufacturing process development was performed to optimize the critical parameters of the ultrafiltration process, which is used to concentrate the antibody to 125 mg/mL concentration, prior to the sterile filtration through two in-line filters and fill-finish operation. During process development, a technical batch with 125 mg/mL product concentration was manufactured.

L-histidine is used in the drug formulation at a concentration of 25 mM as a pH buffer. Sucrose is a stabilizer and also an osmolality regulator (tonicity agent). It can protect the active ingredients from degradation and maintain the osmolality of the drug product. Hydrochloric acid is used as pH adjustment. Water is used as a solvent.

The data obtained up to date demonstrated that the above excipients in the formulation could efficiently maintain the stability of the drug product and do not negatively influence the quality attributes of the drug product.

### Development of Formulation

In the early formulation development phase, several buffer formulations (25 mM glutamate, 25 mM acetate, 25 mM histidine, and 25 mM phosphate) across a pH range (4.5-7.5) and in combination with NaCl (150 mM), sucrose (9%), and polysorbate 80 (0.02%) were tested. Fourteen (14) formulations were prepared and further studied. The following tests were performed during the pre-formulation development phase: visual appearance, protein content, CE-SES (reduced and no-reduced), SEC-HPLC, CEX-HPLC, and DLS.

A preliminary screening study was performed to select formulation conditions for the humanized anti-IGF-1R antibody, making it possible to obtain good stability based on defined analytical criteria.

The purpose of the screening was to perform limited selections of four (4) formulations. The effect of the following 4 factors was studied in the preliminary screening study: (i) the nature of the buffer: 25 mM histidine or 25 mM phosphate; (ii) pH: pH 6-pH 7; (iii) the NaCl concentration: 0-150 mM and (iv) the polysorbate 80 concentration: 0-0.1% v/v.

Two experimental plans (one per buffer) were established to be able to model the response surfaces for each of the plans. The center point was analyzed in triplicate to estimate the experimental variability.

Two different experimental plans were constructed as follows (cubic composite matrices with a center point in triplicate) in two different buffers:
25 mM Histidine buffer experimental plan (refer to Table 1)
25 mM Phosphate buffer experimental plan (refer to Table 2)

TABLE 1
25 mM Histidine buffer experimental plan

| Formula | | | NaCl | Polysorbate 80 |
|---|---|---|---|---|
| No. | Buffer | pH | (mM) | (% v/v) |
| H1 | 25 mM Histidine | 6.0 | 0 | 0 |
| H2 | 25 mM Histidine | 7.0 | 0 | 0 |
| H3 | 25 mM Histidine | 6.0 | 150 | 0 |
| H4 | 25 mM Histidine | 7.0 | 150 | 0 |
| H5 | 25 mM Histidine | 6.0 | 0 | 0.10 |
| H6 | 25 mM Histidine | 7.0 | 0 | 0.10 |
| H7 | 25 mM Histidine | 6.0 | 150 | 0.10 |
| H8 | 25 mM Histidine | 7.0 | 150 | 0.10 |
| H9 | 25 mM Histidine | 6.0 | 75 | 0.05 |
| H10 | 25 mM Histidine | 7.0 | 75 | 0.05 |
| H11 | 25 mM Histidine | 6.5 | 0 | 0.05 |
| H12 | 25 mM Histidine | 6.5 | 150 | 0.05 |
| H13 | 25 mM Histidine | 6.5 | 75 | 0 |
| H14 | 25 mM Histidine | 6.5 | 75 | 0.10 |
| H15 | 25 mM Histidine | 6.5 | 75 | 0.05 |
| H16 | 25 mM Histidine | 6.5 | 75 | 0.05 |
| H17 | 25 mM Histidine | 6.5 | 75 | 0.05 |

TABLE 2
25 mM Phosphate buffer experimental plan

| Formula | | | NaCl | Polysorbate 80 |
|---|---|---|---|---|
| No. | Buffer | pH | (mM) | (% v/v) |
| P1 | 25 mM Phosphate | 6.0 | 0 | 0 |
| P2 | 25 mM Phosphate | 7.0 | 0 | 0 |
| P3 | 25 mM Phosphate | 6.0 | 150 | 0 |
| P4 | 25 mM Phosphate | 7.0 | 150 | 0 |
| P5 | 25 mM Phosphate | 6.0 | 0 | 0.10 |
| P6 | 25 mM Phosphate | 7.0 | 0 | 0.10 |
| P7 | 25 mM Phosphate | 6.0 | 150 | 0.10 |
| P8 | 25 mM Phosphate | 7.0 | 150 | 0.10 |
| P9 | 25 mM Phosphate | 6.0 | 75 | 0.05 |
| P10 | 25 mM Phosphate | 7.0 | 75 | 0.05 |
| P11 | 25 mM Phosphate | 6.5 | 0 | 0.05 |
| P12 | 25 mM Phosphate | 6.5 | 150 | 0.05 |
| P13 | 25 mM Phosphate | 6.5 | 75 | 0 |
| P14 | 25 mM Phosphate | 6.5 | 75 | 0.10 |
| P15 | 25 mM Phosphate | 6.5 | 75 | 0.05 |
| P16 | 25 mM Phosphate | 6.5 | 75 | 0.05 |
| P17 | 25 mM Phosphate | 6.5 | 75 | 0.05 |

The implemented experimental plans were run using specialized software applications (Nemrod and MODDE), which perform a statistical analysis of the data to verify the validity and the relevance of the generated models. These tools make it possible to see the effect of the tested factors on the studied responses by drawing response surfaces and calculating optimums according to defined criteria.

The studied responses for each of the plans were: (i) the value of the melting temperatures Tₘ₁ and Tₘ₂ in ° C. at TO (DSF analysis); (ii) the increase in the rate of acidic variants after 10 days of stress at 40° C. (CIEX analysis) and (iii) the rate of multimers after 10 days of stress at 40° C. (SEC analysis).

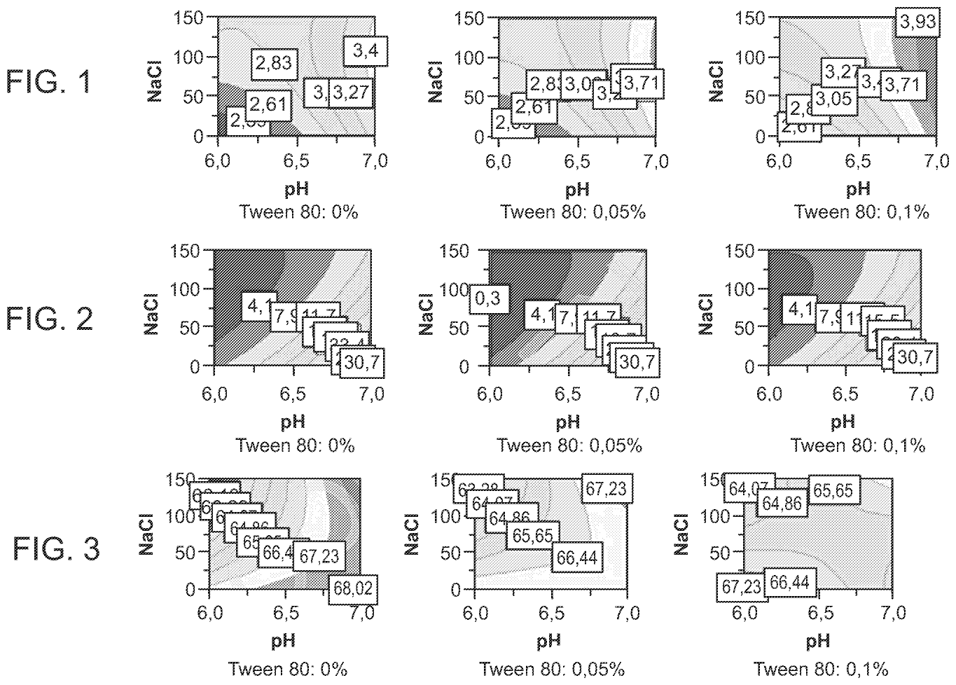

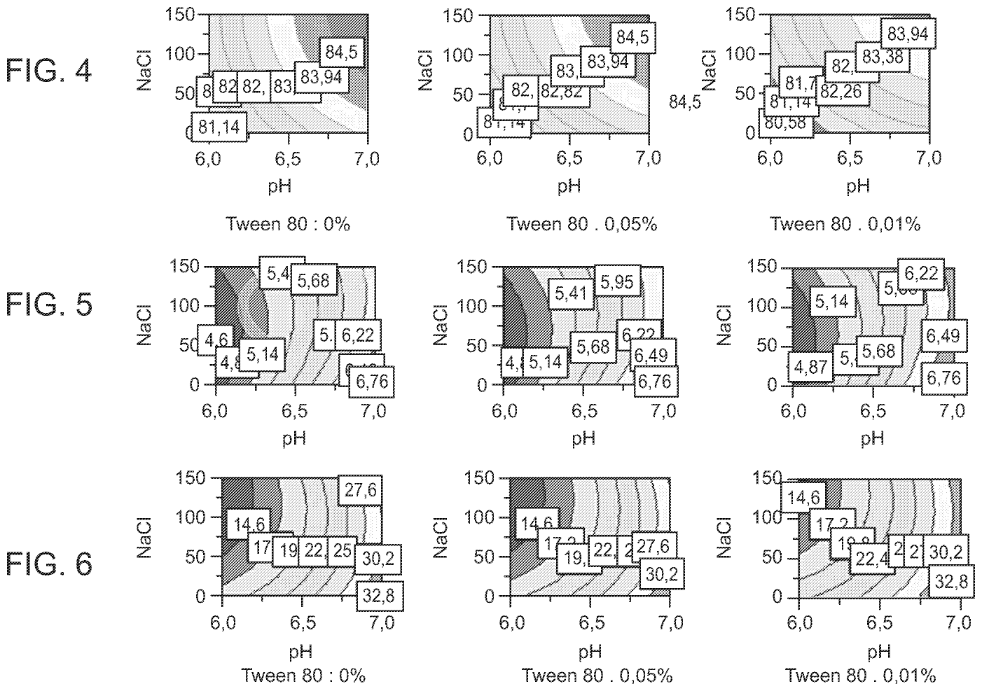

The analysis of the histidine plan showed that the pH has a significant effect on all 3 responses: a high pH (pH 7) causes an increase in the Tm, the rate of multimers and the acidic variants. The presence of NaCl causes an increase in the Tm and the rate of multimers but tends not to cause too many acidic variants to appear. Lastly, the polysorbate 80 does not have too much of an effect on the 3 responses; its presence, at the tested percentage, would even tend to favor the appearance of multimers. The response surfaces are pictured below (FIGS. 1-4).

To minimize the multimer rate, it was preferable to adopt a pH 6-6.5 and salt at <100 mM. The addition of polysorbate 80, at the tested percentage, is not desirable because the presence of the surfactant causes the multimer rate to increase.

To limit the appearance of acidic variants, it was necessary again to adopt a fairly low pH (6-6.5); the presence of NaCl is favorable to stabilize this criterion. However, the presence of polysorbate 80 has no significant effect.

To favor high Tₘ values, pH 7 was preferable and polysorbate 80, at the tested percentage was not desirable.

For the 15 phosphate formulations, the measured Tₘ values are fairly constant: Tₘ₁ only varies between 67.0 and 68.3° C. and Tₘ₂ between 84 and 85° C. The variation of the tested factors does not affect these two criteria. Therefore, it is not relevant to analyze the response surfaces for these two responses.

The analysis of the histidine plan shows that the pH has a significant effect: a high pH (pH 7) causes the multimer rate to increase, as well as the acidic variants. Like histidine, the presence of NaCl tends to cause the multimer rate to increase but limits the appearance of acidic variants. Lastly, polysorbate 80 has no significant effect on the studied responses. The response surfaces are pictured in FIGS. 5 and 6.

To minimize the monomer rate, it was preferable to adopt a pH of 6-6.5. The addition of polysorbate 80, at the tested percentage, is not desirable because the presence of the surfactant increases the multimer rate. The salt does not have a significant effect.

To limit the appearance of acidic variants, it was again necessary to adopt a fairly low pH (6-6.5). As with histidine, the presence of NaCl is favorable for stabilizing this criterion. However, the presence of polysorbate 80 has no significant effect.

The effect of the phosphate buffer factors was very close to the impact of the observed histidine factors (pH 6-6.5, absence of polysorbate 80). However, it is important to note that the levels of multimer rate and appearance of acidic variants are higher in phosphate than in histidine. Minimum and maximum values obtained for the 15 histidine and 15 phosphate formulas are shown in Table 3.

TABLE 3
Minimum and maximum values obtained for the
15 histidine and 15 phosphate formulas

| | Histidine | | Phosphate | |
|---|---|---|---|---|
| | Min | Max | Min | Max |
| Tₘ₁ (° C.) | 63.0 | 68.7 | 67.0 | 68.3 |
| Tₘ₂ (° C.) | 80.0 | 85.0 | 84.0 | 85.0 |
| % Multimers | 2.2 | 4.1 | 4.6 | 7 |
| Appearance acidic variants (%) | 1.7 | 36.3 | 12.5 | 35.1 |

A comprehensive overview was prepared using specialized software applications (Nemrod and MODDE).

Conclusion of the Screening Studies:
(1) It is preferable to formulate the antibody in histidine rather than in phosphate to limit the appearance of multimers and acidic variants;
(2) For histidine, the most favorable pH zone is between pH 6 and pH 6.5;
(3) The presence of polysorbate 80, at the tested percentage, is not desirable; this surfactant is not favorable to stability (based on the studied criteria);
(4) In histidine, the presence of NaCl in the formula is desirable. However, low concentrations (maximum 100 mM) must be favored.

Other elements were considered after carrying out the screening study. The stability monitoring of previously produced antibody lots reveals the appearance of high opalescence (>Standard IV on some lots). This opalescence was not correlated with an evolution in the physicochemical characteristics of the antibody (no degradation or aggregation observed), but it appeared that there was a correlation with the antibody concentration. To limit the opalescence, it appears preferable to decrease the antibody concentration to 10 g/L or even 5 g/L, while the pre-formulation study was done in a worst-case at 20 g/L. The obtained results of selected formulas (P001) are shown in Table 4.

TABLE 4
Opalescence results on lot P001

| Lot | Formula | Opalescence |
|---|---|---|
| P001 a | 25 mM histidine 30 mM NaCl | T0: < I |
| | pH 6.0 (5 g/L) | T 1 month: I-II |
| P001 b | 25 mM histidine 30 mM NaCl | T0: < I |
| | pH 6.0 (10 g/L) | T 1 month: II-III |
| P001 c | 25 mM histidine 100 mM NaCl | T0: < I |
| | pH 6.5 (5 g/L) | T 1 month: I-II |
| P001 d | 25 mM histidine 100 mM NaCl | T0: < I |
| | pH 6.5 (10 g/L) | T 1 month: II-III |

On each of the 4 formulas, no opalescence was observed at To, but the level increases significantly as of 1 month of storage at +4° C.

While lot P001 is not compliant, these results provide insight regarding the relevance of these choices of formulas for the next lot. Therefore, additional tests under stress conditions were performed. Five other formulas with citrate or histidine buffer with or without NaCl were evaluated under stress at 40° C. over 15 days. The presence of NaCl and the antibody concentration appear to increase the opalescence phenomenon. A single formulation stands out for a low level of opalescence: histidine buffer at pH 6. The three citrate formulations whose pH was lower than 6 have a strong opalescence. The analytical monitoring of these five stressed formulations showed that the histidine buffer at pH 6 with 5 g/L protein shows the least impacted charge variant profiles and SEC. The histidine, pH 6, 30 mM NaCl formulation with sucrose at 6% demonstrated low opalescence. The SEC profile also remained acceptable after stress. In order to create diversity in the formulations tested on the next P lot, sucrose was added. Based on all obtained results and considerations, the final four formulations were selected as presented in

Table 5. The selected formulation with 25 mM histidine and 6% sucrose at pH 6.0 was deemed the best performing formulation for the early clinical development.

TABLE 5
Final selection of the 4 formulas for lot P003

| Formula | | Antibody |
|---|---|---|
| code | Composition of the buffer | concentration |
| a | 25 mM histidine pH 6.0 | 5 g/L |
| b | 25 mM histidine pH 6.0 sucrose 6% | 5 g/L |
| c | 25 mM histidine pH 6.0 30 mM NaCl | 5 g/L |
| d | 25 mM histidine pH 6.5 100 mM NaCl | 5 g/L |

### Development of Highly Concentrated Formulation

Additional formulation development studies were performed to enable a highly concentrated solution of the anti-IGF-IR antibody. The formulation containing 25 mM histidine, 5% D-sorbitol, at pH 6.0 was selected for future DS manufacturing.

Four leading formulations at 125 mg/mL were developed, and appropriate testing was conducted. Histidine was used in drug formulations at a concentration of 25 mM as a pH buffering agent, as previously established. Sucrose and D-sorbitol were chosen as osmolality regulators (tonicity agents). Polysorbate 80 (PS80) was included in two of four selected formulations as a surfactant. The composition of the selected formulations is presented Table 6 while the testing plan is included in Table 7.

TABLE 6
Selected compositions of the formulations at 125 mg/mL

| Formulation | | | | | |
|---|---|---|---|---|---|
| No. | pH | Buffer | Sucrose | D-Sorbitol | PS80 |
| 1a | 6.0 | 25 mM L-Histidine | 9% | | |
| 1b | | | 9% | | 0.02% |
| 3a | | | | 5% | |
| 3b | | | | 5% | 0.02% |

TABLE 7
Testing plan in further development of the high dose formulation

| Activity | | | | | | | | |
|---|---|---|---|---|---|---|---|---|
| Thermal Stability Study of VB421 at 125 mg/mL: | | | | | | | | |
| Analytical Timepoints | | | | | | | | Analyses |
| −20° | C. | T0 | | | T4 w | T8 w | T12 w | Visual assessment |
| 2-8° | C. | T0 | | | T4 w | T8 w | T12 w | SEC-HPLC |
| 25° | C. | T0 | | T2 w | T4 w | T8 w | | CEX-HPLC |
| 40° | C. | T0 | T1 w | T2 w | T4 w* | | | DLS |
| 50° | C. | T0 | T1 w | T2 w* | | | | Viscosity (T0 only) |

*Analyses were performed due to acceptable visual assessment

The data generated from the formulation development are summarized below.

After a 12-week duration, samples held at −20° C. and 2-8° C. were removed and assessed visually. All formulations remained clear, with no opalescence, and were a pale amber color. Only a very small number of visible particles were present in some of the samples at −20° C. and 2-8° C. SEC-HPLC results obtained after 12 weeks at −20° C. are shown in Table 8. All formulations displayed no appreciable increase in aggregation, with values for high molecular weight substance (HMWS) comparable to initial tests at T₀. SEC-HPLC results obtained after 12 weeks of storage at 2-8° C. are shown in Table 9. There were no clear differences in stability between formulations at this temperature, only a small increase in HMWS was observed with a difference of around 0.25%. After 2 weeks at 25° C., all formulations showed only a slight reduction in % monomer, with a drop of ˜0.3%, and there were no observable differences in stability between formulations at this temperature and timepoint (Table 10). After 2 weeks at 40° C., there was a small reduction of % monomer by ˜1.1% in all formulations, which was indicative of very good stability, and there was no observable difference in stability between the 4 formulations (Table 11). After 2 weeks at 50° C., there was a more noticeable reduction of % monomer in all formulations, with an overall decrease of about 5.3%. There appeared to be a very slight reduction in stability for formulations 1b and 3b containing PS80 (Table 12). There was no apparent difference between formulation sucrose and sorbitol.

TABLE 8
Thermal stability: −20° C. SEC-HPLC Results

| | | Sum Rel. | Sum Rel. | Rel. Area |
|---|---|---|---|---|
| | Formulation | Area | Area | Monomer |
| | No. | HMWS % | LMWS % | % |
| | DS | 0.53 | 1.13 | 98.34 |
| | reference | | | |
| T₀ | 1a | 0.60 | 1.12 | 98.29 |
| | 1b | 0.60 | 1.08 | 98.33 |
| | 3a | 0.56 | 1.14 | 98.30 |
| | 3b | 0.62 | 1.10 | 98.28 |
| T4w −20° C. | 1a | 0.52 | 1.11 | 98.37 |
| | 1b | 0.53 | 1.09 | 98.39 |
| | 3a | 0.51 | 1.12 | 98.36 |
| | 3b | 0.57 | 1.15 | 98.28 |
| T8w −20° C. | 1a | 0.63 | 1.19 | 98.19 |
| | 1b | 0.64 | 1.21 | 98.15 |
| | 3a | 0.63 | 1.18 | 98.20 |
| | 3b | 0.64 | 1.19 | 98.17 |
| T12w −20° C. | 1a | 0.57 | 1.16 | 98.27 |
| | 1b | 0.64 | 1.19 | 98.17 |
| | 3a | 0.58 | 1.18 | 98.24 |
| | 3b | 0.60 | 1.17 | 98.22 |

TABLE 9
Thermal Stability: 2-8° C. SEC-HPLC Results

| | Formulation | Sum Rel. | Sum Rel. | Rel. Area |
|---|---|---|---|---|
| | No. | Area HMWS % | Area LMWS % | Monomer % |
| | DS reference | 0.53 | 1.13 | 98.34 |
| T0 | 1a | 0.60 | 1.12 | 98.29 |
| | 1b | 0.60 | 1.08 | 98.33 |
| | 3a | 0.56 | 1.14 | 98.30 |
| | 3b | 0.62 | 1.10 | 98.28 |
| T4w | 1a | 0.61 | 1.14 | 98.25 |
| 2-8° C. | 1b | 0.61 | 1.13 | 98.25 |
| | 3a | 0.58 | 1.14 | 98.28 |
| | 3b | 0.62 | 1.17 | 98.22 |
| T8w | 1a | 0.72 | 1.23 | 98.04 |
| 2-8° C. | 1b | 0.75 | 1.20 | 98.06 |
| | 3a | 0.72 | 1.21 | 98.08 |
| | 3b | 0.73 | 1.21 | 98.06 |
| T12w | 1a | 0.69 | 1.25 | 98.06 |
| 2-8° C. | 1b | 0.78 | 1.18 | 98.04 |
| | 3a | 0.70 | 1.21 | 98.09 |
| | 3b | 0.77 | 1.25 | 97.97 |

TABLE 10
Thermal Stability: 25° C. SEC-HPLC Results

| | Formulation | Sum Rel. | Sum Rel. | Rel. Area |
|---|---|---|---|---|
| | No. | Area HMWS % | Area LMWS % | Monomer % |
| | DS reference | 0.53 | 1.13 | 98.34 |
| T0 | 1a | 0.60 | 1.12 | 98.29 |
| | 1b | 0.60 | 1.08 | 98.33 |
| | 3a | 0.56 | 1.14 | 98.30 |
| | 3b | 0.62 | 1.10 | 98.28 |
| T2w | 1a | 0.78 | 1.23 | 97.99 |
| 25° C. | 1b | 0.79 | 1.23 | 97.98 |
| | 3a | 0.77 | 1.26 | 97.97 |
| | 3b | 0.77 | 1.22 | 98.01 |

TABLE 11
Thermal Stability: 40° C. SEC-HPLC Results

| | Formulation | Sum Rel. | Sum Rel. | Rel. Area |
|---|---|---|---|---|
| | No. | Area HMWS % | Area LMWS % | Monomer % |
| | DS reference | 0.53 | 1.13 | 98.34 |
| T0 | 1a | 0.60 | 1.12 | 98.29 |
| | 1b | 0.60 | 1.08 | 98.33 |
| | 3a | 0.56 | 1.14 | 98.30 |
| | 3b | 0.62 | 1.10 | 98.28 |
| T1w | 1a | 0.98 | 1.32 | 97.70 |
| 40° C. | 1b | 1.00 | 1.36 | 97.63 |
| | 3a | 0.94 | 1.38 | 97.69 |
| | 3b | 0.96 | 1.34 | 97.69 |
| T2w | 1a | 1.13 | 1.71 | 97.16 |
| 40° C. | 1b | 1.14 | 1.74 | 97.12 |
| | 3a | 1.09 | 1.73 | 97.18 |
| | 3b | 1.08 | 1.71 | 97.22 |

TABLE 12
Thermal Stability: 50° C. SEC-HPLC Results

| | Formulation | Sum Rel. | Sum Rel. | Rel. Area |
|---|---|---|---|---|
| | No. | Area HMWS % | Area LMWS % | Monomer % |
| | DS reference | 0.53 | 1.13 | 98.34 |
| T0 | 1a | 0.60 | 1.12 | 98.29 |
| | 1b | 0.60 | 1.08 | 98.33 |
| | 3a | 0.56 | 1.14 | 98.30 |
| | 3b | 0.62 | 1.10 | 98.28 |
| T1w | 1a | 1.78 | 2.88 | 95.34 |
| 50° C. | 1b | 1.90 | 3.16 | 94.94 |
| | 3a | 1.69 | 2.99 | 95.31 |
| | 3b | 1.76 | 3.21 | 95.03 |
| T2w | 1a | 2.53 | 4.47 | 93.00 |
| 50° C. | 1b | 2.65 | 4.67 | 92.68 |
| | 3a | 2.27 | 4.74 | 92.99 |
| | 3b | 2.25 | 4.79 | 92.96 |

CEX-HPLC analysis revealed that the formulations showed good chemical stability after 12 weeks at-20° C. (Table 13) and 2-8° C. (Table 14). From the CEX-HPLC data, slightly higher values for % main species were observed for samples kept at 2-8° C. This indicated that the freeze-thaw cycles undergone by samples at −20° C. did not affect aggregation but were more pronounced for the chemical stability of the molecule.

TABLE 13
Thermal stability: −20° C. CEX-HPLC Results

| | | Sum Rel. | Sum Rel. | Rel. Area |
|---|---|---|---|---|
| | Formulation | Area Acidic | Area | Main Peak |
| | No. | Sp. % | Basic Sp. % | % |
| | DS | 27.77 | 8.23 | 64.00 |
| | reference | | | |
| T0 | 1a | 28.58 | 8.01 | 63.41 |
| | 1b | 28.46 | 8.04 | 63.51 |
| | 3a | 27.93 | 8.35 | 63.72 |
| | 3b | 28.24 | 7.97 | 63.79 |
| T4w −20° C. | 1a | 28.32 | 8.35 | 63.33 |
| | 1b | 28.32 | 8.23 | 63.45 |
| | 3a | 27.67 | 8.20 | 64.14 |
| | 3b | 27.93 | 8.30 | 63.77 |
| T8w −20° C. | 1a | 28.22 | 10.21 | 61.57 |
| | 1b | 28.29 | 10.22 | 61.48 |
| | 3a | 27.98 | 9.64 | 62.38 |
| | 3b | 28.02 | 9.94 | 62.04 |
| T12w −20° C. | 1a | 28.23 | 10.18 | 61.58 |
| | 1b | 28.19 | 10.10 | 61.72 |
| | 3a | 27.69 | 9.91 | 62.40 |
| | 3b | 27.75 | 10.24 | 62.01 |

TABLE 14
Thermal Stability: 2-8° C. CEX-HPLC Results

| | | Sum Rel. Area | Sum Rel. Area | Rel. Area Main |
|---|---|---|---|---|
| | Formulation | Acidic Sp. | Basic Sp. | Peak |
| | No. | % | % | % |
| | DS reference | 27.77 | 8.23 | 64.00 |
| T0 | 1a | 28.58 | 8.01 | 63.41 |
| | 1b | 28.46 | 8.04 | 63.51 |
| | 3a | 27.93 | 8.35 | 63.72 |
| | 3b | 28.24 | 7.97 | 63.79 |
| T4w | 1a | 27.33 | 8.31 | 64.36 |
| 2-8° C. | 1b | 28.01 | 8.15 | 63.83 |
| | 3a | 27.89 | 7.84 | 64.27 |
| | 3b | 27.94 | 8.10 | 63.96 |
| T8w | 1a | 28.24 | 9.82 | 61.93 |
| 2-8° C. | 1b | 28.38 | 9.80 | 61.82 |
| | 3a | 27.97 | 9.76 | 62.28 |
| | 3b | 27.99 | 9.87 | 62.14 |
| T12w | 1a | 28.14 | 9.92 | 61.94 |
| 2-8° C. | 1b | 28.24 | 9.84 | 61.92 |
| | 3a | 27.56 | 9.78 | 62.66 |
| | 3b | 27.84 | 9.90 | 62.25 |

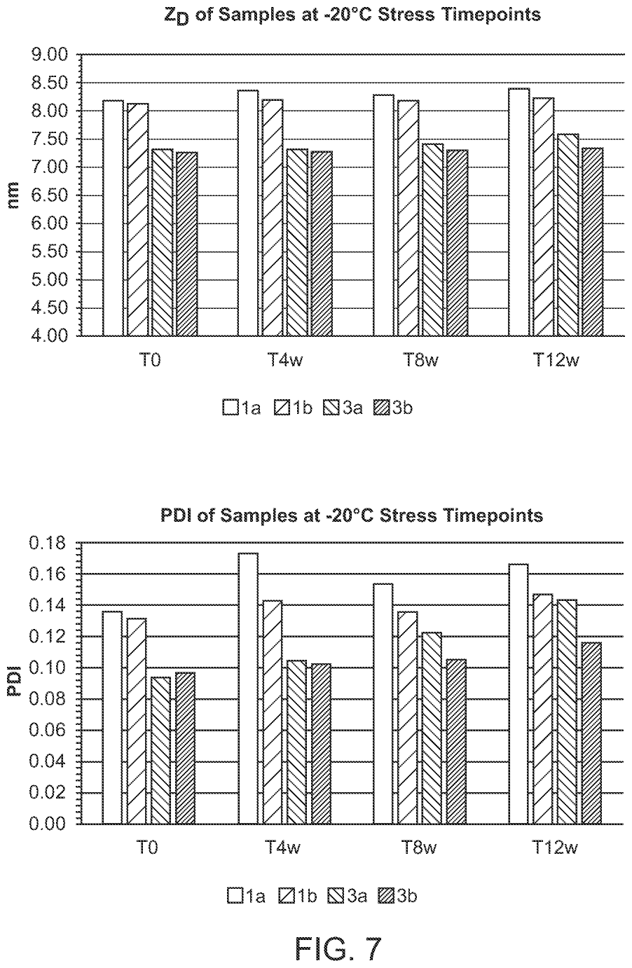

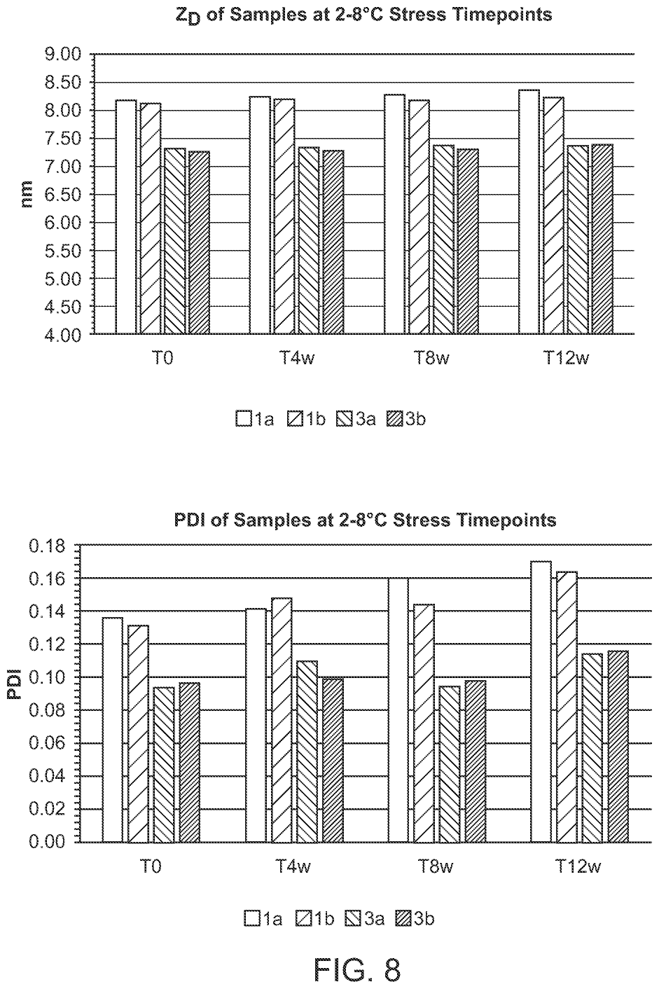

DLS measurements revealed that particle size ZD was virtually unchanged after 12 weeks at both temperatures. PDI values show a trend towards increasing values over the timepoints, but all formulations remain monodisperse after 12 weeks (FIG. 7 and FIG. 8).

### Conclusion:

The obtained results concluded that formulation 3a, which contained 25 mM histidine and sorbitol as tonicity modifier without PS80, offered marginally better stability than other formulations. The formulation containing 25 mM histidine and sucrose demonstrated a similar profile to the formulation with sorbitol. Therefore, the Drug Substance formulated in 25 mM histidine, 6% sucrose, at pH 6.0, which was originally selected for the drug substance at 20 mg/mL, was deemed safe and acceptable for the manufacturing of the clinical product at 125 mg/mL.

Establishment of the Manufacturing Process for the Antibody High Dose Drug Product (125 mg/mL)

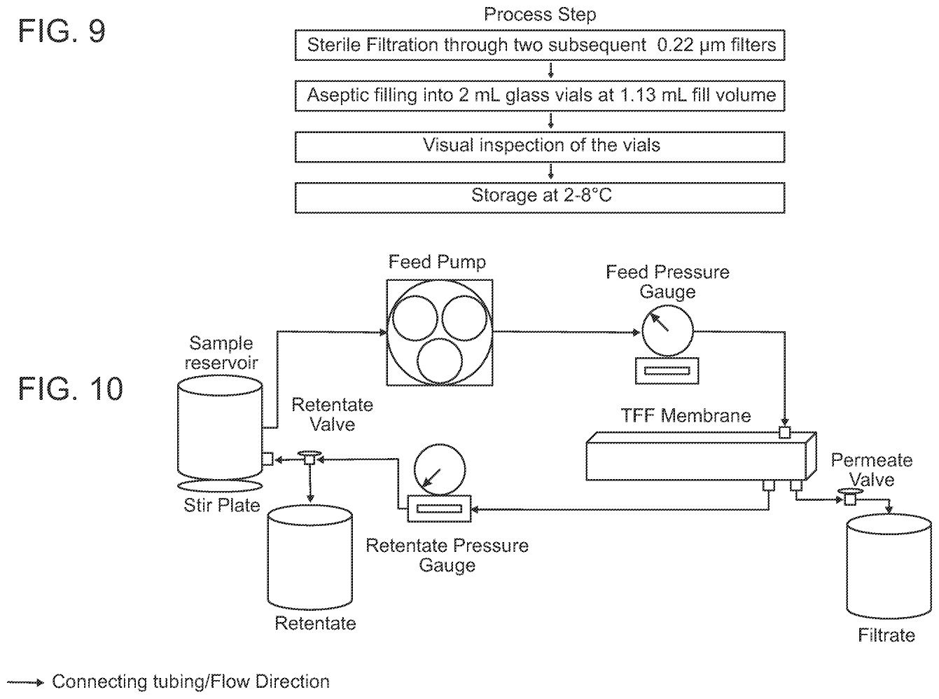

The drug product manufacturing process for the low dose drug product for clinical Phase 1 shown in FIG. 9. The antibody drug substance (20 mg/mL) is thawed at 2-8° C. Then the formulation is sterile filtered, filled into vials, and sealed.

In order to concentrate drug substance to 125 mg/mL prior to manufacturing of the high dose drug product, a tangential flow filtration (TFF) step is performed. TFF also called as a cross-flow filtration system, is a rapid and efficient method used to concentrate solutions by removing fluids while keeping the solute molecules. This process is done by selecting a filter or membrane having a pore size significantly smaller than the solute molecules to allow for the retention of solute molecules. Pall's Minimate EVO system with pump, pressure gauge, retentate screw clamp, reservoirs, and tubing connections is used in the process.

The concentration process was developed at small scale using the bulk drug substance from the GMP batch as discussed below.

PES membrane with a molecular weight cut-off (MWCO) of 30 kDa was used to concentrate the drug product solution from 20 mg/mL to a target concentration of 125 mg/mL. The TFF process flow diagram is presented in FIG. 10.

The process of concentration of the drug product solution started with a preparation of formulation buffer containing histidine and sucrose. The formulation buffer was used to pre-condition the TFF membranes before the addition of the sample. Pre-conditioning was used to remove air bubbles and equilibrate the system.

After buffer pre-conditioning, the thawed drug substance solution (20 mg/mL) was added and mixed using the peristaltic pump at a flow rate of about 40 mL/min. The drug substance solution was concentrated to a final volume of 18-20 mL in the container. After the retentate was collected, the system was flushed with the formulation buffer that was added to the concentrated product intermediate. The concentrated product intermediate was filtered through a 0.4 μm-pre-filter followed by two 0.22 μm PVDF filters. The filtered solution was aliquoted into 2R Class 1 glass vials and stoppered. This technical batch's process parameters and the components used in the manufacturing were representative of that proposed for the manufacturing of the 125 mg/mL clinical batch. The technical batch was tested using the same methods as used for clinical DP batches' release, and it was placed on stability, as a representative batch, to generate the stability data prior to manufacturing of the GMP batch.

### In-Use Compatibility

The antibody drug product (DP) is supplied as a solution for subcutaneous injection (SC) and intravenous infusion (IV). For administration by IV infusion, the antibody is diluted with saline solution, while no dilution is needed for SC administration. The in-use compatibility study demonstrated good stability of the diluted drug product to 2.1 mg/mL and 0.07 mg/mL in saline at various holding conditions and infusion rates:
Ambient temperature for up to 24 h, fast infusion rate at 100 mL/h
Ambient temperature for up to 24 h, slow infusion rate at 50 mL/h
Refrigerated conditions for 24 h followed by holding at ambient temperature for an additional 24 h, fast infusion rate at 100 mL/h
Refrigerated conditions for 24 h followed by holding at ambient temperature for an additional 24 h, slow infusion rate at 50 mL/h

The in-use compatibility study confirmed that the drug product does not adhere to the bag and/or tubing, and that there is no aggregation and/or materials loss during the filtration phase. The infusion set used in the in-use compatibility study was representative of the infusion set proposed for the clinical study. Therefore, the antibody is compatible with the selected infusion system. The in-use compatibility study brackets the proposed infusion parameters, as defined in the Pharmacy Manual and supports accurate dosing of the antibody drug product in the clinic.

Transparent, ethylene vinyl acetate (EVA) infusion bags (100 mL) equipped with three connection points (supplied by ICU Medical P/N SN2010BP) and Pump/gravity Clearlink set with in-line BCV, the vented chamber with 15-micron filter, ClickLuer, SafePrime, line label (supplied by Baxter International Inc, P/N BXISMMC96901L) were used in the study. The drug substance, batch T102 formulated in the histidine and sucrose formulation was tested. The T102 DS was qualified as a reference standard

Two concentrations of diluted solution, 2.1 mg/mL and 0.07 mg/mL in saline solution, were tested. The total volume after dilution is greater than 100 mL to allow the bulk solution to be charged into two infusion bags for each storage condition. To samples were removed and analyzed in duplicate for both concentrations at each storage condition.

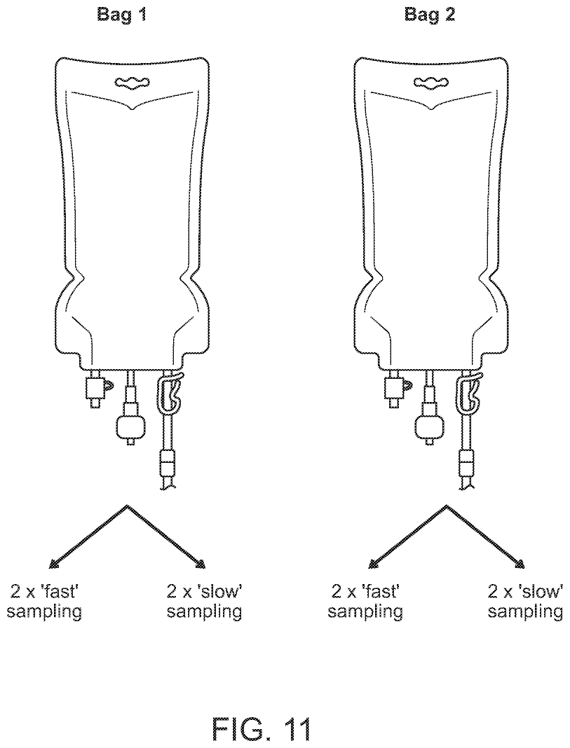

For compatibility studies, a fill volume of 50 mL, 50% of a maximum volume of the infusion bag, was selected to mimic the worst-case scenario with large surface area contact. The lowest concentration in the bag was considered the worst-case scenario due to a higher surface-to-protein exposure ratio. The infusion bags were stored at room temperature as well as at 2-8° C. After planned storage, the infusion bags were withdrawn and connected to the rest of the infusion set, and solutions were passed through the infusion line/filter at an infusion rate of 100 mL/1 hour (“fast”) and 50 mL/1 hour (“slow”). After the infusion process completion, the samples were analyzed. For each infusion rate, two samples were taken. A representative diagram is provided in FIG. 11 to indicate the “fast” and “slow” ports to which other infusion devices described above were connected.

For infusion bags held at room temperature, samples were analyzed after two hours (Timepoint (T)=2 h), four hours (T=4 h), and twenty-four hours (T=24 h).

For infusion bags held at 2-8° C.: The bags were held at 2-8° C. for 24 h. At the end of the storage period, the infusion bags were removed from the refrigerator to equilibrate to ambient temperature for 2 h. Samples were taken at the end of the 2 h equilibration period for analysis (T=2 h), after additional two hours (T=4 h), and after twenty-four hours since being moved out of the refrigerator (T=24 h). For all samples, the test items included appearance, protein content by A₂₈₀, SEC-HPLC, and CEX-HPLC. The methods were run with system suitability to ensure the credibility of the results.

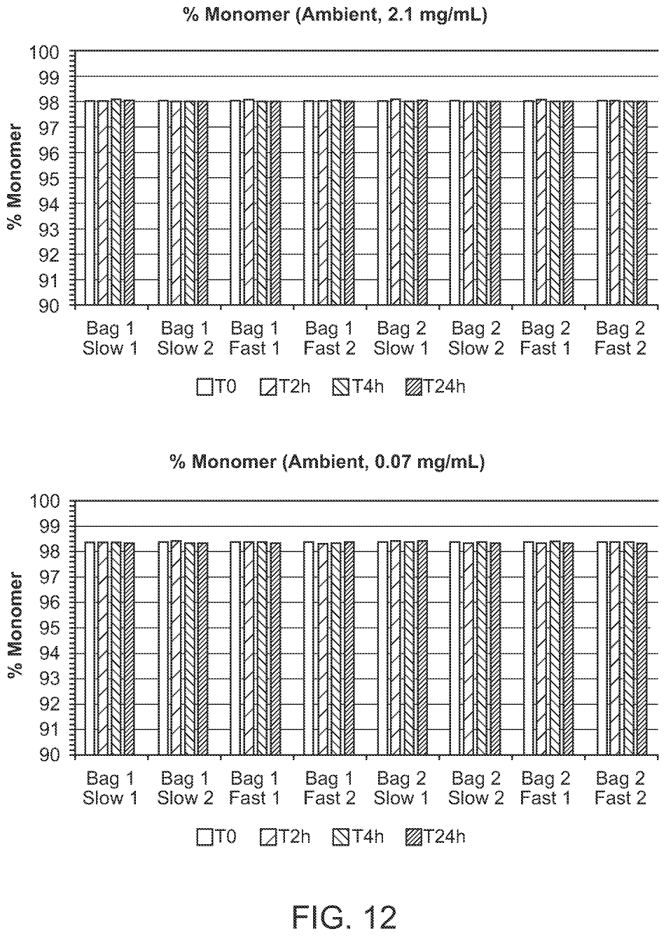

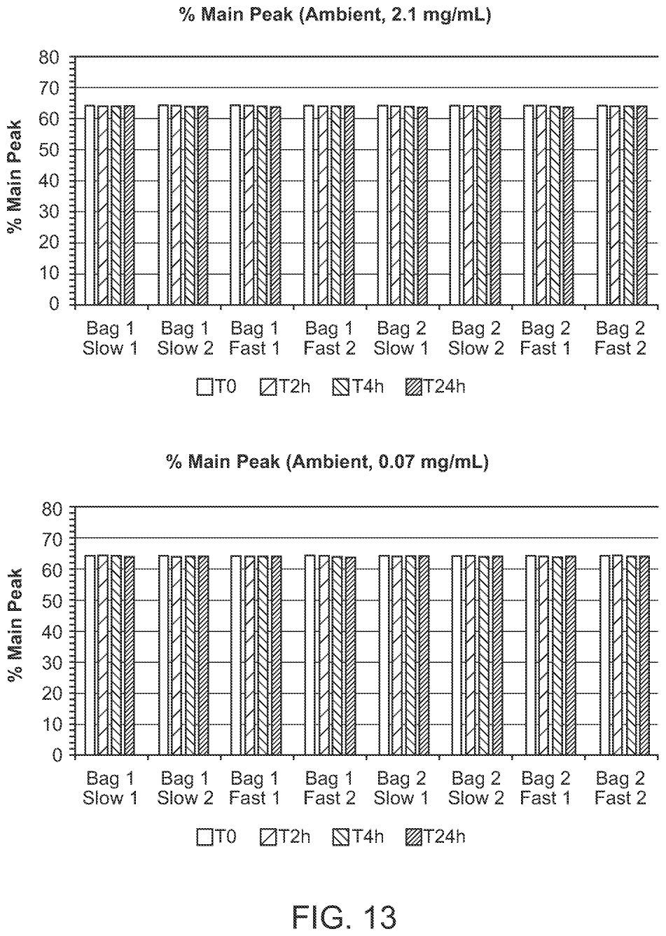

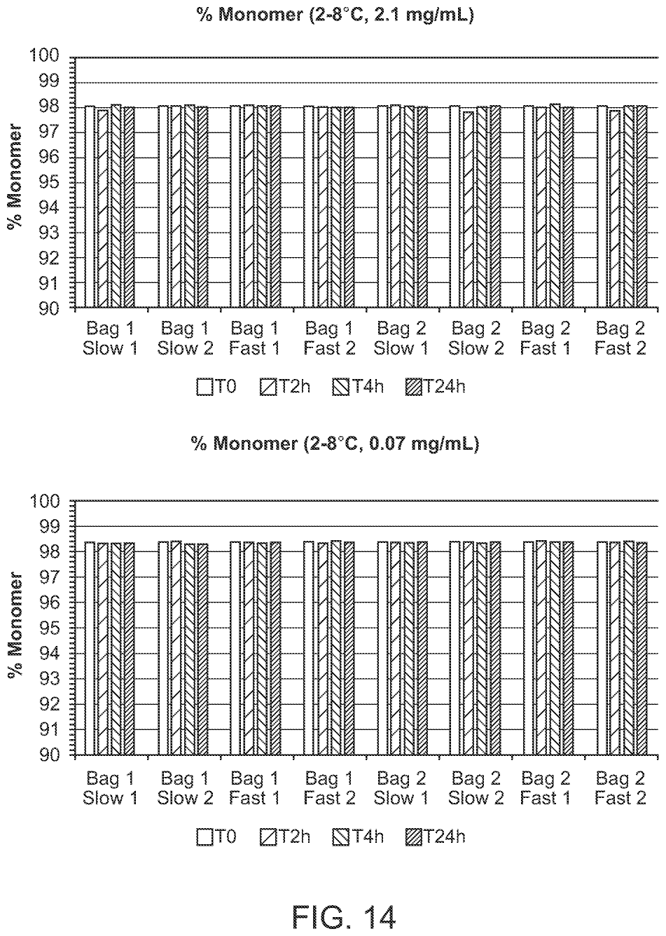

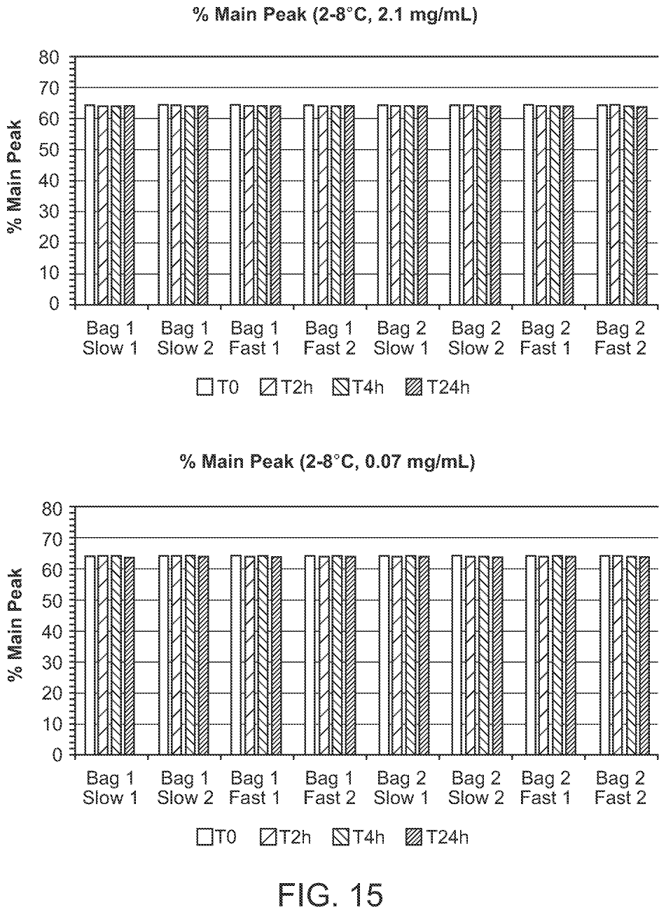

The results for appearance and protein content in the in-use compatibility study are presented in Table 15 to Table 22. The trends for the monomer content by SEC-HPLC and main form content by CEX-HPLC are shown in FIGS. 12-15. All four quality attributes of diluted antibody drug product exhibited excellent stability throughout the storage in the infusion bag at both ambient and refrigerated temperatures and after passing through the infusion lines and filters at the fast and slow infusion rates.

TABLE 15
Results for In-Use Compatibility Study at Room Temperature,
2.1 mg/mL in Saline and Infusion Rate of 100 mL/1 hour

| | Time Point | | | |
|---|---|---|---|---|
| Tests | 0 h¹ | 2 h | 4 h | 24 h |

Appearance

| | | | | |
|---|---|---|---|---|
| Bag 1 | Clear, | Clear, | Clear, | Clear, |
| | colorless, no | colorless, no | colorless, no | colorless, no |
| | visible | visible | visible | visible |
| | particles | particles | particles | particles |
| Bag 2 | Clear, | Clear, | Clear, | Clear, |
| | colorless, no | colorless, no | colorless, no | colorless, no |
| | visible | visible | visible | visible |
| | particles | particles | particles | particles |

Protein content by UV (mg/mL)

| | | | | | |
|---|---|---|---|---|---|
| Bag 1 | Fast 1 | 2.28 | 2.25 | 2.28 | 2.31 |
| | Fast 2 | | 2.24 | 2.29 | 2.31 |
| Bag 2 | Fast 1 | | 2.25 | 2.32 | 2.33 |
| | Fast 2 | | 2.24 | 2.30 | 2.30 |

¹Average of the sample taken at T0 that was analyzed in duplicate

TABLE 16
Results for In-Use Compatibility Study at Room Temperature,
0.07 mg/mL in Saline and Infusion Rate of 100 mL/1 hour

| | Time Point | | | |
|---|---|---|---|---|
| Tests | 0 h | 2 h | 4 h | 24 h |

Appearance

| | | | | |
|---|---|---|---|---|
| Bag 1 | Clear, | Clear, | Clear, | Clear, |
| | colorless, no | colorless, no | colorless, no | colorless, no |
| | visible | visible | visible | visible |
| | particles | particles | particles | particles |
| Bag 2 | Clear, | Clear, | Clear, | Clear, |
| | colorless, no | colorless, no | colorless, no | colorless, no |
| | visible | visible | visible | visible |
| | particles | particles | particles | particles |

Protein content by UV (mg/mL)

| | | | | | |
|---|---|---|---|---|---|
| Bag 1 | Fast 1 | 0.08 | 0.06 | 0.09 | 0.11 |
| | Fast 2 | | 0.07 | 0.11 | 0.10 |
| Bag 2 | Fast 1 | | 0.06 | 0.11 | 0.11 |
| | Fast 2 | | 0.07 | 0.10 | 0.11 |

TABLE 17
Results for In-Use Compatibility Study at Room Temperature,
2.1 mg/mL in Saline and Infusion Rate of 50 mL/1 hour

| | Time Point | | | |
|---|---|---|---|---|
| Tests | 0 h | 2 h | 4 h | 24 h |

Appearance

| | | | | |
|---|---|---|---|---|
| Bag 1 | Clear, | Clear, | Clear, | Clear, |
| | colorless, no | colorless, no | colorless, no | colorless, no |
| | visible | visible | visible | visible |
| | particles | particles | particles | particles |
| Bag 2 | Clear, | Clear, | Clear, | Clear, |
| | colorless, no | colorless, no | colorless, no | colorless, no |
| | visible | visible | visible | visible |
| | particles | particles | particles | particles |

Protein content by UV (mg/mL)

| | | | | | |
|---|---|---|---|---|---|
| Bag 1 | Slow 1 | 2.28 | 2.25 | 2.30 | 2.30 |
| | Slow 2 | | 2.26 | 2.28 | 2.32 |
| Bag 2 | Slow 1 | | 2.23 | 2.29 | 2.31 |
| | Slow 2 | | 2.24 | 2.27 | 2.31 |

TABLE 18
Results for In-Use Compatibility Study at Room Temperature,
0.07 mg/mL in Saline and Infusion Rate of 50 mL/1 hour

| | Time Point | | | |
|---|---|---|---|---|
| Tests | 0 h | 2 h | 4 h | 24 h |

Appearance

| | | | | |
|---|---|---|---|---|
| Bag 1 | Clear, | Clear, | Clear, | Clear, |
| | colorless, no | colorless, no | colorless, no | colorless, no |
| | visible | visible | visible | visible |
| | particles | particles | particles | particles |
| Bag 2 | Clear, | Clear, | Clear, | Clear, |
| | colorless, no | colorless, no | colorless, no | colorless, no |
| | visible | visible | visible | visible |
| | particles | particles | particles | particles |

Protein content by UV (mg/mL)

| | | | | | |
|---|---|---|---|---|---|
| Bag 1 | Slow 1 | 0.08 | 0.05 | 0.11 | 0.09 |
| | Slow 2 | | 0.06 | 0.10 | 0.11 |

TABLE 19
Results for In-Use Compatibility Study at 2-8° C.,
2.1 mg/mL in Saline and Infusion Rate of 100 mL/1 hour

| | Time Point | | | |
|---|---|---|---|---|
| Tests | 0 h¹ | 2 h | 4 h | 24 h |

Appearance

| | | | | |
|---|---|---|---|---|
| Bag 1 | Clear, | Clear, | Clear, | Clear, |
| | colorless, no | colorless, no | colorless, no | colorless, no |
| | visible | visible | visible | visible |
| | particles | particles | particles | particles |
| Bag 2 | Clear, | Clear, | Clear, | Clear, |
| | colorless, no | colorless, no | colorless, no | colorless, no |
| | visible | visible | visible | visible |
| | particles | particles | particles | particles |

Protein content by UV (mg/mL)

| | | | | | |
|---|---|---|---|---|---|
| Bag 1 | Fast 1 | 2.22 | 2.19 | 2.25 | 2.26 |
| | Fast 2 | | 2.19 | 2.26 | 2.26 |
| Bag 2 | Fast 1 | | 2.21 | 2.27 | 2.26 |
| | Fast 2 | | 2.20 | 2.22 | 2.26 |

¹Average of the sample taken at T0 that was analyzed in duplicate

TABLE 20
Results for In-Use Compatibility Study at 2-8° C.,
0.07 mg/mL in Saline and Infusion Rate of 100 mL/1 hour

| | Time Point | | | |
|---|---|---|---|---|
| Tests | 0 h | 2 h | 4 h | 24 h |

Appearance

| | | | | |
|---|---|---|---|---|
| Bag 1 | Clear, | Clear, | Clear, | Clear, |
| | colorless, no | colorless, no | colorless, no | colorless, no |
| | visible | visible | visible | visible |
| | particles | particles | particles | particles |
| Bag 2 | Clear, | Clear, | Clear, | Clear, |
| | colorless, no | colorless, no | colorless, no | colorless, no |
| | visible | visible | visible | visible |
| | particles | particles | particles | particles |

Protein content by UV (mg/mL)

| | | | | | |
|---|---|---|---|---|---|
| Bag 1 | Fast 1 | 0.07 | 0.08 | 0.10 | 0.10 |
| | Fast 2 | | 0.08 | 0.11 | 0.10 |
| Bag 2 | Fast 1 | | 0.07 | 0.11 | 0.10 |
| | Fast 2 | | 0.08 | 0.11 | 0.09 |

TABLE 21
Results for In-Use Compatibility Study at 2-8° C.,
2.1 mg/mL in Saline and Infusion Rate of 50 mL/1 hour

| | Time Point | | | |
|---|---|---|---|---|
| Tests | 0 h | 2 h | 4 h | 24 h |

Appearance

| | | | | |
|---|---|---|---|---|
| Bag 1 | Clear, | Clear, | Clear, | Clear, |
| | colorless, no | colorless, no | colorless, no | colorless, no |
| | visible | visible | visible | visible |
| | particles | particles | particles | particles |
| Bag 2 | Clear, | Clear, | Clear, | Clear, |
| | colorless, no | colorless, no | colorless, no | colorless, no |
| | visible | visible | visible | visible |
| | particles | particles | particles | particles |

Protein content by UV (mg/mL)

| | | | | | |
|---|---|---|---|---|---|
| Bag 1 | Slow 1 | 2.22 | 2.20 | 2.26 | 2.26 |
| | Slow 2 | | 2.18 | 2.24 | 2.26 |
| Bag 2 | Slow 1 | | 2.21 | 2.30 | 2.26 |
| | Slow 2 | | 2.19 | 2.27 | 2.26 |

TABLE 22
Results for In-Use Compatibility Study at 2-8° C.,
0.07 mg/mL in Saline and Infusion Rate of 50 mL/1 hour

| | Time Point | | | |
|---|---|---|---|---|
| Tests | 0 h | 2 h | 4 h | 24 h |

Appearance

| | | | | |
|---|---|---|---|---|
| Bag 1 | Clear, | Clear, | Clear, | Clear, |
| | colorless, no | colorless, no | colorless, no | colorless, no |
| | visible | visible | visible | visible |
| | particles | particles | particles | particles |
| Bag 2 | Clear, | Clear, | Clear, | Clear, |
| | colorless, no | colorless, no | colorless, no | colorless, no |
| | visible | visible | visible | visible |
| | particles | particles | particles | particles |

Protein content by UV (mg/mL)

| | | | | | |
|---|---|---|---|---|---|
| Bag 1 | Slow 1 | 0.07 | 0.07 | 0.11 | 0.10 |
| | Slow 2 | | 0.07 | 0.10 | 0.10 |
| Bag 2 | Slow 1 | | 0.07 | 0.10 | 0.10 |
| | Slow 2 | | 0.07 | 0.09 | 0.08 |

### Example 2: High Concentration Formulation Development and Injectability Study

The high concentration behaviour of the antibody is investigated in the lead buffer selected from the previous stages:
Buffer 3a: 25 mM L-histidine, 5% w/v D-sorbitol, pH 6.0.
Concentrate VB421 to 150, 175 and 200 mg/mL (125 mg/mL will also be tested, although the stability as this concentration is understood).

Stability at these concentrations is assessed by visual appearance, SEC and DLS. Viscosity is also measured, and an injectability assessment is preformed to ensure that injection forces are not too high.

### Buffer Exchange:

Sample was loaded into Sartorius VivaSpin® 20 (20 mL Capacity), 30 kDa MWCO, PES membrane (DS details: 20 mg/mL, F50085, Hz208F2-4, G101D). 5 cycles of buffer exchange were performed.

### Concentration:

Samples were concentrated to 125, 150, 175 and 200 mg/mL. Concentrations were confirmed by protein A280 measurement.

### Observations:

As before, no issues were encountered during concentration; with no formation of visible fibrils/particles or clogging of the filter membrane.

### Appearance Method

Vials are gently swirled and viewed under an off-white light source (10W LED, 2000-3750 Lux). Vials are observed against both black and white backgrounds.

At the elevated concentrations the only noticeable visual effect was a slightly darker amber colour than observed at 125 mg/mL. All samples were free of opalescence and visible aggregates.

### SEC-HPLC Method

Samples are analysed at 1 mg/mL (diluted in mobile phase). Injection Volume is 10 μL. Buffer is 150 mM sodium phosphate, pH 6.9. Run: Isocratic, 0.35 mL/min, 15 min. Column temperature: Ambient. Column: Agilent AdvanceBio SEC Column (p/n 1580-5301). HPLC System: Dionex Ultimate-3000 UHPLC system. Detection Wavelength: 280 nm. Samples injected in duplicate—average results reported.

There was a very small increase (0.2%) in the amount of HMWS in concentrating the DS from 20 mg/mL. There were no further increases in HMWS between 125 and 200 mg/mL, and the monomer % was also similar. LMWS were ˜1.5% for all concentrations.

### Zetasizer Method:

System: Zetasizer Ultra. Cell: ZEN2112, Low Volume Quartz Batch Cuvette. Measurement Temperature: 25° C. Equilibration Time: 120 Seconds. Data Processing: General Purpose. Angle of detection: Back Scatter. Attenuation: Automatic. Measurement Process: Automatic. No pause after sub runs. No Optical Filter. Pause between repeats: 0 seconds. 1×Replicates; 5×Measurements per replicate. Sample prep: Centrifugation and filtration through 0.22 μm filter. Samples were analysed neat, sample volume ˜50 μL.

There was a non-significant increase in the z-average (ZD) at higher concentrations. The PDI also increased at the higher concentrations, but still remained <0.2, indicating a high level of monodispersity. It is interesting to note that the lowest Z_{D} and PDI values were seen for 150 mg/mL.

### Viscosity Measurements

Viscosity was measured on a RheoSense m-VROC viscometer. Measurements were recorded at 21° C. Rate sweeps were performed for the injectability assessment by varying the shear rates between 10-90% of the range of the chip. The viscosity at 150 mg/mL was slightly lower than at 125 mg/mL, this is in agreement with the DLS results, which showed a similar trend.

### Injectability Assessment

Many high-concentration therapeutics exhibit non-Newtonian (shear-thinning) behaviour-meaning that the viscosity is reduced at high shear rates. During injection through a 27G needle, shear rates >100000 s⁻¹ can be reached at injection rates of 0.1 mL/s. If injectability is only evaluated from low shear rate viscosity measurements, higher-than-expected injection forces could be calculated. This could lead to potential formulations of interest being removed from further consideration based an incorrect injectability assessment.

The injection force for non-Newtonian fluids can be calculated as follows:

$${F}_{v} ≅ 2 π {l}_{n} {σ}_{w} \frac{{R}_{p}^{2}}{{R}_{n}}$$

where:

Fᵥ=injection force (N), lₙ=needle length (m), σ_{w}=shear stress at the wall of the needle (Pa), calculated by plotting the shear stress and shear rate. Rₚ=inner radius of the syringe (m). Rₙ=inner radius of the needle (m).

Once the force (Fᵥ—in N) has been calculated, this can be used as an approximate guide as to how easy a fluid is to inject. A recent study (Robinson et al., Adv. Healthcare. Mater., 2020, 9, 1901521) suggests the following: Where Fᵥ<12 N, injection takes minimal effort. Where Fᵥ=12-38 N, effort is required. Where Fᵥ=38-64 N, great effort is required. Where Fᵥ>64 N, injection is not possible.

This method only estimates the viscousity's contribution to the injection force of the fluid in the syringe. The friction forces are not considered. The friction force varies and is dependent on the syringe piston and barrel materials. The calculation does not factor in the effect of lubrication of the barrel and piston from the drug within the syringe. Additionally, the resistance of injecting fluids into SC tissue is not considered.

Varying shear rates from 300-100000 s⁻¹ caused a drop in viscosity of ˜1 cP. There was a linear relationship between shear rate and shear stress.

At 125 mg/mL, the viscosities were relatively low (9.5-8.5 cP). The injection force was calculated for a variety of needle/syringe combinations. The preferred combination is underlined in Table 23, and gives an injection force well within the desired range (<12 N). A “26s” needle is overall the same outer diameter as a regular 26G needle, but has a smaller inner diameter and thicker walls to improve durability, this results in a significantly higher injection force for this variant.

TABLE 23
Injectability assessment results at 125 mg/ml.

| | Needle | Syringe | Piston | Injection | Injection |
|---|---|---|---|---|---|
| Needle | Length | Size | Diameter | Rate | Force |
| Gauge | (mm) | (mL) | (mm) | (mm³/s) | (N) |
| 21G | 12.7 | 1 | 4.78 | 20 | 0.01 |
| | | | | 100 | 0.03 |
| | | | | 200 | 0.06 |
| 25G | 12.7 | 1 | 4.78 | 20 | 0.09 |
| | | | | 100 | 0.45 |
| | | | | 200 | 0.90 |
| 26G | 12.7 | 3 | 8.66 | 20 | 0.30 |
| | | | | 100 | 1.47 |
| | | | | 200 | 2.94 |
| 26G | 12.7 | 1 | 4.78 | 20 | 0.09 |
| | | | | 100 | 0.45 |
| | | | | 200 | 0.90 |
| 27G | 12.7 | 1 | 4.78 | 20 | 0.21 |
| | | | | 100 | 1.05 |
| | | | | 200 | 2.10 |
| 30G | 12.7 | 1 | 4.78 | 20 | 0.63 |
| | | | | 100 | 3.11 |
| | | | | 200 | 6.16 |

Varying shear rates from 300-100000 s⁻¹ caused a drop in viscosity of ˜1 cP. The viscosity seemed to be lower at 150 mg/mL. There was a linear relationship between shear rate and shear stress. At 150 mg/mL, non-Newtonian behaviour was observed at the higher shear rates. As with the 125 mg/mL samples, the only syringe/needle combinations giving potential injectability issues were with the 26s needle.

TABLE 24
Injectability assessment results at 150 mg/ml.

| | Needle | Syringe | Piston | Injection | Injection |
|---|---|---|---|---|---|
| Needle | Length | Size | Diameter | Rate | Force |
| Gauge | (mm) | (mL) | (mm) | (mm³/s) | (N) |
| 21G | 12.7 | 1 | 4.78 | 20 | 0.01 |
| | | | | 100 | 0.03 |
| | | | | 200 | 0.05 |
| 25G | 12.7 | 1 | 4.78 | 20 | 0.08 |
| | | | | 100 | 0.38 |
| | | | | 200 | 0.75 |
| 26G | 12.7 | 3 | 8.66 | 20 | 0.25 |
| | | | | 100 | 1.24 |
| | | | | 200 | 2.46 |
| 26G | 12.7 | 1 | 4.78 | 20 | 0.08 |
| | | | | 100 | 0.38 |
| | | | | 200 | 0.75 |
| 27G | 12.7 | 1 | 4.78 | 20 | 0.18 |
| | | | | 100 | 0.88 |
| | | | | 200 | 1.75 |
| 30G | 12.7 | 1 | 4.78 | 20 | 0.53 |
| | | | | 100 | 2.60 |
| | | | | 200 | 5.13 |

Varying shear rates from 250-100000 s⁻¹ caused a drop in viscosity of ˜2.5 cP. There was a linear relationship between shear rate and shear stress. At 175 mg/mL, non-Newtonian behaviour was observed at the higher shear rates. Again, the only samples 5 showing potential issues were with the 26s needles.

TABLE 25
Injectability assessment results at 175 mg/ml.

| | Needle | Syringe | Piston | Injection | Injection |
|---|---|---|---|---|---|
| Needle | Length | Size | Diameter | Rate | Force |
| Gauge | (mm) | (mL) | (mm) | (mm³/s) | (N) |
| 21G | 12.7 | 1 | 4.78 | 20 | 0.01 |
| | | | | 100 | 0.04 |
| | | | | 200 | 0.09 |
| 25G | 12.7 | 1 | 4.78 | 20 | 0.13 |
| | | | | 100 | 0.64 |
| | | | | 200 | 1.28 |
| 26G | 12.7 | 3 | 8.66 | 20 | 0.42 |
| | | | | 100 | 2.12 |
| | | | | 200 | 4.21 |
| 26G | 12.7 | 1 | 4.78 | 20 | 0.13 |
| | | | | 100 | 0.64 |
| | | | | 200 | 1.28 |
| 27G | 12.7 | 1 | 4.78 | 20 | 0.30 |
| | | | | 100 | 1.51 |
| | | | | 200 | 2.99 |
| 30G | 12.7 | 1 | 4.78 | 20 | 0.90 |
| | | | | 100 | 4.43 |
| | | | | 200 | 8.71 |

Varying shear rates from 250-100000 s⁻¹ caused a drop in viscosity of ˜2.5 cP. There was a linear relationship between shear rate and shear stress. At 200 mg/mL, non-Newtonian behaviour was observed at the higher shear rates. Again, the only samples showing potential issues were with the 26s needles.

TABLE 26
Injectability assessment results at 200 mg/ml.

| | Needle | Syringe | Piston | Injection | Injection |
|---|---|---|---|---|---|
| Needle | Length | Size | Diameter | Rate | Force |
| Gauge | (mm) | (mL) | (mm) | (mm³/s) | (N) |
| 21G | 12.7 | 1 | 4.78 | 20 | 0.01 |
| | | | | 100 | 0.05 |
| | | | | 200 | 0.10 |
| 25G | 12.7 | 1 | 4.78 | 20 | 0.15 |
| | | | | 100 | 0.73 |
| | | | | 200 | 1.44 |
| 26G | 12.7 | 3 | 8.66 | 20 | 0.48 |
| | | | | 100 | 2.38 |
| | | | | 200 | 4.74 |
| 26G | 12.7 | 1 | 4.78 | 20 | 0.15 |
| | | | | 100 | 0.73 |
| | | | | 200 | 1.44 |
| 27G | 12.7 | 1 | 4.78 | 20 | 0.34 |
| | | | | 100 | 1.70 |
| | | | | 200 | 3.36 |
| 30G | 12.7 | 1 | 4.78 | 20 | 1.01 |
| | | | | 100 | 4.98 |
| | | | | 200 | 9.76 |

### Injectability Assessment—Conclusions

At higher concentrations, more significant non-Newtonian behaviour was observed. The antibody appeared to be injectable under most conditions tested. At 200 mg/mL the injection force for a rate of 200 mm³/s was <10 N. The preferred combination of a 3 mL syringe and 26G needle gave calculated injection forces of 2.5-4.7 N which are all lower than the suggested value of 12 N, at which value effort is required to inject fluids.

The antibody was concentrated to 200 mg/mL without any detriment to visual appearance, and remained free of opalescence and visible aggregates throughout the concentration steps. SEC and DLS did not change significantly between 125 and 200 mg/mL. Viscosity did increase, especially at 175 and 200 mg/mL, however values were still 14-16 cP. Injectability of the antibody indicated an injection force of <5 N at all concentrations, suggesting low effort being required to inject this composition.

### Example 3: Deamidation and Oxidation Analysis

The deamidation and oxidation analysis was performed on the samples in Table 27.

TABLE 27
Samples for deamidation and oxidation analysis.

| Sample | | Concentration |
|---|---|---|
| No. | Sample ID | (mg/mL) |
| 1 | VAIO-1799_1a: 25 mM histidine, 9% w/v | 125 |
| | sucrose, pH 6.0 | |
| 2 | VAIO-1799_1b: 25 mM histidine, 9% w/v | 125 |
| | sucrose, 0.02% w/v PS80, pH 6.0 | |
| 3 | VAIO-1799_3a: 25 mM histidine, 5% w/v | 125 |
| | sorbitol, pH 6.0 | |
| 4 | VAIO-1799_3b: 25 mM histidine, 5% w/v | 125 |
| | sorbitol, 0.02% w/v PS80, pH 6.0 | |

TABLE 28
Details of the formulations samples.

| | | | Molecular Weight Standards | | | |
|---|---|---|---|---|---|---|
| | | | 155.15 | 342.3 | 182.17 | N/A |
| | | | Stock Concentration | | | |
| | | | 250 mM | 56 | 40 | 4 |
| | | | Supplier/Grade | | | |
| | | | Merck | Merck | Sigma | Merck |
| | | | Emprove ® | Emprove ® | Aldrich ≥98% | Emprove ® |
| | | | Part number | | | |
| | | | 104352 | 100892 | S1876 | 817061 |
| Formulation | pH | Units API conc | mM | % (w/v) | % (w/v) | % (w/v) |
| 1a | 6 | 125 | 25 | 9 | | |
| 1b | 6 | 125 | 25 | 9 | | 0.02 |
| 3a | 6 | 125 | 25 | | 5 | |
| 3b | 6 | 125 | 25 | | 5 | 0.02 |

Sample preparation: Buffer exchange into Tris-HCl pH 8, Digestion using Immobilized Trypsin (Smart Digest, Thermo Scientific), Reduction with DTT.

RP-ESI-MSe Analysis: System: Waters Nano Aquity UPLC in line with a Xevo G2S QTOF; Injection volume: 2 μL (1 μg of IgG); Separation: RP on a Waters Acquity UPLC BEH300 C18 1.7 μm 2.1×100 mm column. Buffer A: 0.1% FA in Water, Buffer B: 0.1% FA in Acetonitrile; Elution with linear gradient 3% to 50% B; Mass spectrometry: positive ion mode, MSe scan 250-2000 m/z.

PTMs identification criteria used for UNIFI™ and further manual investigation: Exact mass within 20 ppm for intact peptide, Fragment ions pattern for confirming identity, Clear resolved isotope pattern.

Deamidations were only identified in HC. In between N and Q deamidation identified only at N and majority of the fragments' ions containing either N or Q were not found deamidated. No second deamidation (2×) was found on any of the deamidated peptide both in HC an LC.

TABLE 29
List of fragments of the antibody and their deamination.

| | | | | | % | %Response |
|---|---|---|---|---|---|---|
| Fragment | | | Sequence | Sequence | Response | 1x |
| Label | Peptide | Modifiers | start | end | Native | Deamidation |
| HC:T5 | QAPGQGLEWMG | | 39 | 59 | 100 | |
| | WIWPGDGSTK | | | | | |
| | (SEQ ID NO: 16) | | | | | |
| HC:T10 | SEDTAVYFCASP | Deamidation | 88 | 124 | 96.9 | 3.1 |
| | MITPNYAMDYW | N | | | | |
| | GQGTLVTVSSAS | | | | | |
| | TK | | | | | |
| | (SEQ ID NO: 17) | | | | | |
| HC:T13 | DYFPEPVTVSWN | | 151 | 213 | 100 | |
| | SGALTSGVHTFP | | | | | |
| | AVLQSSGLYSLS | | | | | |
| | SVVTVPSSSLGT | | | | | |
| | QTYICNVNHKPS | | | | | |
| | NTK | | | | | |
| | (SEQ ID NO: 18) | | | | | |
| HC:T21 | FNWYVDGVEVH | Deamidation | 278 | 291 | 99.5 | 0.5 |
| | NAK | N | | | | |
| | (SEQ ID NO: 19) | | | | | |
| HC:T23 | EEQYNSTYR | Glycosylation | 296 | 304 | 100 | |
| | (SEQ ID NO: 20) | G0F N | | | | |
| HC:T24 | VVSVLTVLHQD | Deamidation | 305 | 320 | 197.6 | 2.4 |
| | WLNGK | N | | | | |
| | (SEQ ID NO: 21) | | | | | |
| HC:T32 | EPQVYTLPPSR | | 348 | 358 | 100 | |
| | SEQ ID NO: 22) | | | | | |
| HC:T34 | NQVSLTCLVK | Deamidation | 364 | 373 | 99.2 | 0.8 |
| | (SEQ ID NO: 23) | N | | | | |
| HC:T35 | GFYPSDIAVEWE | Deamidation | 374 | 395 | 99.8 | 0.2 |
| | SNGQPENNYK | N | | | | |
| | (SEQ ID NO: 24) | | | | | |
| HC:T39 | WQQGNVFSCSV | Deamidation | 420 | 442 | 99.8 | 0.2 |
| | MHEALHNHYTQ | N | | | | |
| | K | | | | | |
| | (SEQ ID NO: 25) | | | | | |
| LC:T1 | DIQMTQSPSSLSA | | 1 | 18 | 100 | |
| | SVGDR | | | | | |
| | (SEQ ID NO: 26) | | | | | |
| LC:T4 | YLNWYQQKPGK | | 32 | 42 | 100 | |
| | SEQ ID NO: 27) | | | | | |
| LC:T7 | LQSGVPSR | | 54 | 61 | 100 | |
| | (SEQ ID NO: 28) | | | | | |
| LC:T9 | GSGTDYSLTISSL | | 66 | 103 | 100 | |
| | QPEDFATYFCQQ | | | | | |
| | GSTLPYTFGGGT | | | | | |
| | K | | | | | |
| | (SEQ ID NO: 29) | | | | | |
| LC:T12 | TVAAPSVFIFPPS | | 109 | 126 | 100 | |
| | DEQLK | | | | | |
| | (SEQ ID NO: 30) | | | | | |
| LC:T13 | SGTASVVCLLNN | | 127 | 142 | 100 | |
| | FYPR | | | | | |
| | (SEQ ID NO: 31) | | | | | |
| LC:T15 | VQWK | | 146 | 149 | 100 | |
| | (SEQ ID NO: 32) | | | | | |
| LC:T16 | VDNALQSGNSQE | | 150 | 169 | 100 | |
| | SVTEQDSK | | | | | |
| | (SEQ ID NO: 33) | | | | | |
| LC:T20 | VYACEVTHQGLS | | 191 | 207 | 100 | |
| | SPVTK | | | | | |
| | (SEQ ID NO: 34) | | | | | |

Deamidation is only identified at HC. In between N and Q deamidation identified only in N and majority of the fragments' ions containing either N or Q were not found deamidated. At HC: T35 (2×) N residue of the peptide was found deamidated (second deamidation indicated as 2×).

Oxidation was identified at both M and W residues of the fragments ions but majority of the fragment ions containing M and W residues were found not oxidised. At HC: T24 two W residues of the peptide chain were found oxidised (second oxidation indicated as 2×). At LC: T1 1× oxidation was also observed.

TABLE 30
List of fragments of the antibody and their oxidation.

| | | | | | | 1x | 2x |
|---|---|---|---|---|---|---|---|
| Fragment | | Sequence | Sequence | b/y | Native | Oxidation | Oxidation |
| label | Peptide | start | end | Ions | % | % | % |
| HC:T5 | QAPGQGLEWMG | 39 | 59 | 3 | 100.0 | 2.8 | |
| | WIWPGDGSTK | | | | | | |
| | (SEQ ID NO: 35) | | | | | | |
| HC:T8 | VTMTR | 68 | 72 | 2 | 100.0 | | |
| | (SEQ ID NO: 36) | | | | | | |
| HC:T9 | DTSTSTVYMELS | 73 | 87 | 9 | 100.0 | | |
| | SLR | | | | | | |
| | (SEQ ID NO: 37) | | | | | | |
| HC:T10 | SEDTAVYFCASP | 88 | 124 | 5 | 84.6 | 16.4 | |
| | MITPNYAMDYW | | | | | | |
| | GQGTLVTVSSAS | | | | | | |
| | TK | | | | | | |
| | (SEQ ID NO: 38) | | | | | | |
| HC:T13 | DYFPEPVTVSWN | 151 | 213 | 1 | 100.0 | | |
| | SGALTSGVHTFP | | | | | | |
| | AVLQSSGLYSLS | | | | | | |
| | SVVTVPSSSLGT | | | | | | |
| | QTYICNVNHKPS | | | | | | |
| | NTK | | | | | | |
| | (SEQ ID NO: 39) | | | | | | |
| HC:T19 | DTLMISR | 252 | 258 | 7 | 84.9 | 15.1 | |
| | (SEQ ID NO: 40) | | | | | | |
| HC:T21 | FNWYVDGVEVH | 278 | 291 | 15 | 99.9 | | 0.1 |
| | NAK | | | | | | |
| | (SEQ ID NO: 41) | | | | | | |
| HC:T24 | VVSVLTVLHQD | 305 | 320 | 15 | 99.3 | | 0.7 |
| | WLNGK | | | | | | |
| | (SEQ ID NO: 42) | | | | | | |
| HC:T35 | GFYPSDIAVEWE | 374 | 395 | 22 | 100.0 | | |
| | SNGQPENNYK | | | | | | |
| | (SEQ ID NO: 43) | | | | | | |
| HC:T39 | WQQGNVFSCSV | 420 | 442 | 16 | 97.7 | 3.3 | |
| | MHEALHNHYTQ | | | | | | |
| | K | | | | | | |
| | (SEQ ID NO: 44) | | | | | | |
| LC:T1 | DIQMTQSPSSLSA | 1 | 18 | 7 | 98.6 | 1.4 | |
| | SVGDR | | | | | | |
| | (SEQ ID NO: 45) | | | | | | |
| LC:T4 | YLNWYQQKPGK | 32 | 42 | 1 | 100.0 | | |
| | (SEQ ID NO: 46) | | | | | | |
| LC:T15 | VQWK | 146 | 149 | 0 | 100.0 | | |
| | (SEQ ID NO: 47) | | | | | | |

Oxidation was identified at both M and W residues of the fragments ions. At HC: T24 two W residues of the peptide chain were found oxidised (second oxidation indicated as 2×). At LC no oxidation was observed.

The PTM of the VAIO-1799 samples analysed using deamidation on Q and N and Oxidation at M and W. Peptides were resolved by Reversed phase LC-MS and identified by exact mass and fragment sequence tags. Deamidation is identified at N however, oxidation identified at both M and W. Increased deamidation observed in 1a and 3a compared to 1b and 3b respectively. Increase oxidation observed in 1b compared to 1a (particularly on HC-88-124). Similarly increased oxidation observed in 3a compared to 3b.

### Example 4: Safety, Tolerability, and Pharmacokinetics

A Phase I trial was conducted to evaluate the safety, tolerability, pharmacokinetics, and pharmacodynamics (PD) of single ascending doses (SAD) of IV or SC lonigutamab in healthy participants. The study conducted was a single-center, randomized, double-blind, placebo-controlled, sequential single ascending dose (SAD) study. The study involved eight (8) cohorts of healthy volunteers, which were divided into two groups: four (4) cohorts receiving intravenous administration of lonigutamab and four (4) cohorts receiving subcutaneous administration of lonigutamab.

Specifically, cohorts 1˜4 received intravenous administration of 0.1 mg/kg, 0.3 mg/kg, 1.0 mg/kg, or 3.0 mg/kg of lonigutamab or matching placebo over the course of 60 minutes. Cohorts 5-8 received fixed-does subcutaneous injections dose administered in a 2 mL or less volume at doses of 20 mg, 40 mg, 125 mg, or 250 mg of lonigutamab or matching placebo.

The participants in the study were healthy as per the study protocol. Specifically, the participants were aged between 18 and 55 years old, and had a body-mass index (BMI) in the range of 18-32 kg/m² when they were enrolled in the study.

In the course of the study, routine laboratory, audiology, pharmacokinetic, and pharmacodynamic assessments were performed at pre-determined time points in each of the cohort involved in the study. In addition, safety and tolerability of the administered drug were evaluated through the course of study through the assessment of adverse events and were monitored over 99 days.

The pharmacokinetic data was obtained using a validated electrochemiluminescent immunoassay. The receptor occupancy was evaluated using a qualified flow cytometry assay.

The study involved enrollment of sixty-three (63) participants. In these participants, forty-seven (47) received lonigutamab and sixteen (16) received placebo. In this study, four (4) participants discontinued because they were lost in follow up or other reasons. This loss of participants were not deemed not to impact the primary analyses.

The baseline characteristics and demographics of the participants are provided in Table 31 below.

TABLE 31
Baseline Characteristics:

| | Safety Population | |
|---|---|---|
| | (n = 63) | |
| Age (years) | 35.7 | (19-55) |
| Sex | | |
| Male | 48 | (76.2) |
| Female | 15 | (23.8) |
| Race | | |
| American Indian or Alaska Native | 1 | (1.6) |
| Black/African American | 34 | (54) |
| White | 23 | (36.5) |
| Unknown | 3 | (4.8) |
| Multiple | 2 | (3.2) |
| Weight (kg) | 78.2 | (52.9-100.1) |
| BMI (kg/m²) | 25.67 | (20.0-29.6) |

The data in Table 31 is provided in n (%) or mean (range). There were no meaningful differences across treatment groups for any of the characteristics.

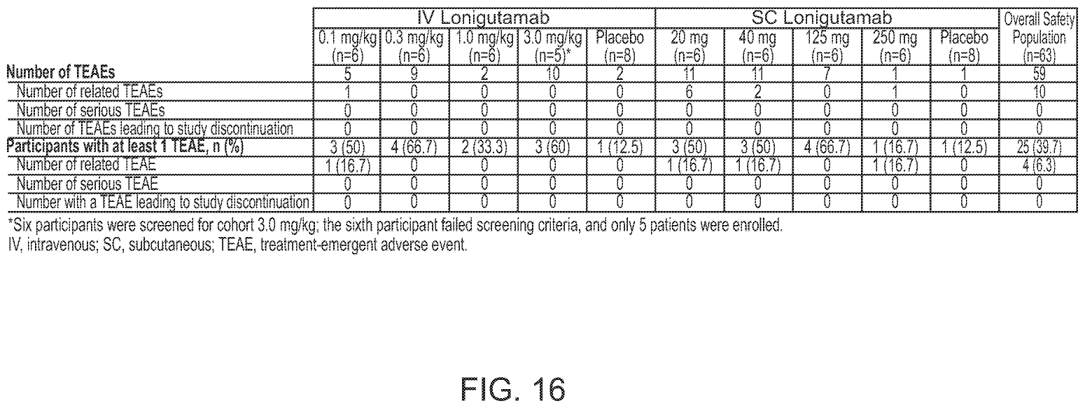

The preliminary data for safety and tolerability assessment obtained by tracking the treatment-emergent adverse events (TEAEs) is presented in FIG. 16.

The data demonstrates that no serious adverse events (SAEs) occurred. All the adverse events were mild and moderate in severity, and dose-related safety events occurred. Overall, the target-related adverse events of special interest (AESI) that were closely monitored for included allergic reactions type I/anaphylaxis, hyperglycemia, infusion-related reactions, diarrhea/inflammatory bowel disease exacerbation, hearing impairment, and muscle spasms. In the study, one (1.6%) AESI was observed in one (1) participant; gastrointestinal disorders (abdominal pain, abdominal distention, and frequent bowel movements) that were “possibly” or “probably” related to lonigutamab (participant reported more than 1 TEAE) in the 20 mg SC cohort.

One participant had an infusion-related reaction and three (3) had injection site reactions that were “possibly” or “probably” related to lonigutamab. All reactions were mild and self-limited, resolving within a few hours without medical intervention.

### Pharmacokinetic Assessments:

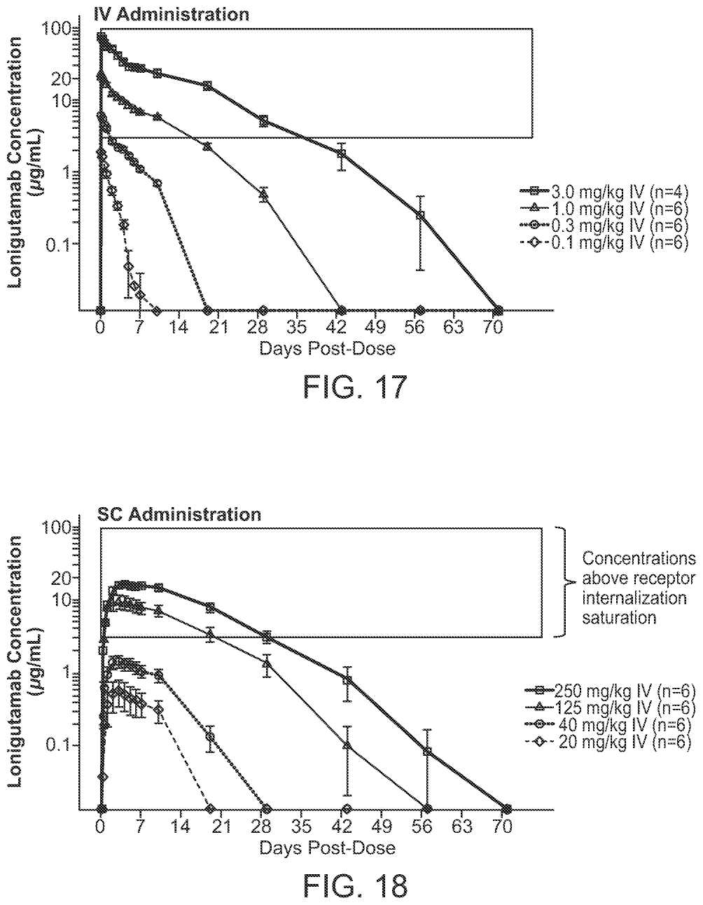

The mean serum lonigutamab concentration over time profiles following intravenous (IV) administration is presented in FIG. 17. The mean serum lonigutamab concentration over time profiles following subcutaneous (SC) administration is presented in FIG. 18.

In the data provided in FIGS. 17 and 18, samples below the limit of quantification (0.1 μg/mL) are plotted as 0 μg/mL in the 3.0 mg/kg, n does not include 1 participant who discontinued early in the study due to deployment, as previously noted. The shaded area in FIG. 17 and FIG. 18 represents the serum concentrations of lonigutamab, which were above the IGF-IR internalization saturation.

As evident from the data provided in FIG. 17 and FIG. 18, mean serum concentrations of lonigutamab increased in a greater than dose proportional manner over the dose ranges tested. Moreover, FIG. 17 and FIG. 18 also provide indication of target-mediated drug disposition (TMDD) after the administration of IV and/or SC administration of lonigutamab. TMDD describes a nonlinear pharmacokinetics (PK) phenomenon that is caused by high-affinity binding of a compound to its pharmacologic targets. Once lonigutamab binds to IGF-1R, the resulting complex is degraded. At low concentrations, the serum concentration of lonigutamab declines rapidly as lonigutamab efficiently binds to IGF-1R and is degraded, and thus, there is a rapid decline in the measured serum concentrations of lonigutamab. In contrast, at high concentrations, the target, IGF-1R is saturated with lonigutamab, and the serum concentrations of lonigutamab declines slowly. This slow decline of serum concentrations of lonigutamab indicates that it is available to bind to newly generated IGF-1R. In addition to the pharmacology of lonigutamab, this data provides additional evidence of IGF-1R internalization.

This data demonstrates that TMDD threshold, above which IGF-IR internalization is saturated, appears to be around 3 μg/mL lonigutamab. As compared to IV administration, SC administration of lonigutamab overcomes TMDD to maintain pharmacologically relevant concentrations of lonigutamab.

### Pharmacodynamic Assessments:

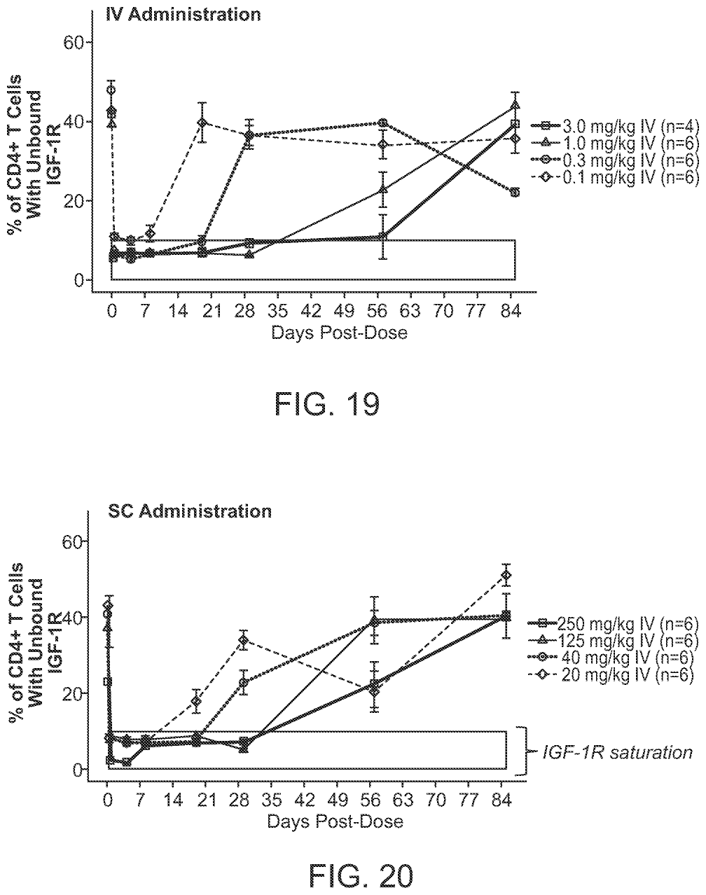

The mean lonigutamab receptor occupancy on peripheral blood mononuclear cells over time following intravenous administration is provided in FIG. 19. The mean lonigutamab receptor occupancy on peripheral blood mononuclear cells over time following subcutaneous administration is provided in FIG. 20. The shaded area in FIG. 19 and FIG. 20 demonstrates the area of optimal IGF-IR internalization after the administration of the pharmaceutical composition comprising lonigutamab.

As noted in the data provided in FIGS. 19 and 20, maximal IGF-IR occupancy was observed by the first timepoint (12 hours) in all cohorts. The duration of receptor saturation increased with increasing dose of lonigutamab. The 1.0 mg/kg and 3.0 mg/kg (administered as an intravenous infusion) and 125 mg and 250 mg (administered as a subcutaneous injection) were maintained at a maximal level of receptor occupancy for a period of at least 4 weeks. As noted previously, in the 3.0 mg/kg cohort, n does not include one (1) participant who discontinued early in the study. The shaded area in FIGS. 19 and 20 indicates receptor saturation.

### Example 5: Multiple Dose Trial for Subcutaneous Administration

In a planned trial, 36 participants with thyroid eye disease (TED) will be enrolled for multicenter, double-masked, placebo-controlled, randomized, multiple ascending dose (MAD) clinical study to evaluate lonigutamab versus placebo. This study will involve up to 3 cohorts, wherein each cohort receives three (3) different treatment arms.

The proposed eligibility criteria for participation in this study is provided below:
Ages Eligible for Study: 18 Years to 65 Years (Adult, Older Adult)
Sexes Eligible for Study: All
Criteria
Key Inclusion Criteria:
   Male or female, ≥18 and ≤65 years of age.
   Proptosis defined in the study eye as ≥3 mm above normal.
   Clinical Activity Score (CAS)≥4 (using a 7-item scale) for the most severely affected eye.
   Onset of active TED symptoms within 24 months prior to the baseline.
   Must agree to use highly effective contraception as specified in the protocol
Key Exclusion Criteria:
   Biopsy-proven or clinically suspected inflammatory bowel disease or irritable bowel syndrome.
   Clinically significant pathology related to hearing
   Corneal decompensation unresponsive to medical management.
   Previous orbital irradiation (for any cause) or any previous surgical treatment for TED.
   Subjects with diabetes or hemoglobin A1c>6.0%.
   Any steroid use (intravenous [IV] or oral) with a cumulative dose equivalent to >3 g of methylprednisolone for the treatment of TED.
   Previous steroid use (IV or oral) specifically for the treatment of TED not to exceed 1 g total dose in the 8 weeks prior to Screening.
   Previous use of teprotumumab or any other IGF-1 receptor (IGF-1R) inhibitor.
   Any previous treatment with a biologic drug for the treatment of TED (eg, rituximab and tocilizumab).
Cohort 1 will receive a single subcutaneous injection of 20 mg lonigutamab or placebo on days 1 and 21.
Cohort 2 will receive a single subcutaneous injection of 125 mg lonigutamab or placebo on days 1 and 21.
Cohort 3 will receive a single subcutaneous injection of 250 mg lonigutamab or placebo on days 1 and 21.

The primary outcomes to be measured in the study will include incidence and characterization of nonserious treatment emergent adverse events (TEAEs) between days 1 and 113. The outcomes measured will also include incidence and characterization of serious TEAEs between days 1 and 113.

The secondary outcomes measured will include:
1. PK profile of lonigutamab [Time Frame: Day 1 to Day 113]
   Area under the concentration-time curve from time zero to the infinity (AUCO-inf)
2. PK profile of lonigutamab [Time Frame: Day 1 to Day 113]
   Maximum observed concentration (Cmax)
3. PK profile of lonigutamab [Time Frame: Day 1 to Day 113]
   Time of observed Cmax (Tmax)
4. PK profile of lonigutamab [Time Frame: Day 1 to Day 113]
   Area under the concentration-time curve from time zero to the last quantifiable concentration (AUC_{O}-last)
5. PK profile of lonigutamab [Time Frame: Day 1 to Day 113]
   Elimination half-life (T1/2 el)
6. PK profile of lonigutamab [Time Frame: Day 1 to Day 113]
   Elimination rate constant (Kel)
7. PK profile of lonigutamab [Time Frame: Day 1 to Day 113]
   Total body clearance (CL/F)
8. PK profile of lonigutamab [Time Frame: Day 1 to Day 113]
   Volume of distribution (Vz/F)
9. Proportion of subjects that develop anti-drug antibodies (ADAs) after administration of multiple doses of lonigutamab [Time Frame: Day 1 to Day 113]
   Pharmacokinetic and Pharmacodynamic data

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A pharmaceutical composition comprising:
(a) at least 75 mg/ml of an anti-IGF-IR antibody comprising a CDRH1 of SEQ ID NO: 1, a CDRH2 of SEQ ID NO: 2, a CDRH3 of SEQ ID NO: 3, a CDRL1 of SEQ ID NO: 4, a CDRL2 of SEQ ID NO: 5, and a CDRL3 of SEQ ID NO: 6;
(b) from 20 to 30 mM histidine; and
(c) from 4% to 6% D-sorbitol;
wherein the pharmaceutical composition is at a pH of 5.5-6.5.

2. The pharmaceutical composition of claim 1, wherein the antibody comprises a heavy chain variable domain comprising an amino acid sequence of SEQ ID NO: 7.

3. The pharmaceutical composition of claim 1 or 2, wherein the antibody comprises a light chain variable domain comprising an amino acid sequence of SEQ ID NO: 8.

4. The pharmaceutical composition of claim 1, wherein the antibody comprises a heavy chain comprising an amino acid sequence of SEQ ID NO: 9, SEQ ID NO: 11, SEQ ID NO: 12, or SEQ ID NO: 13.

5. The pharmaceutical composition of claim 1 or 4, wherein the antibody comprises a light chain comprising an amino acid sequence of SEQ ID NO: 10.

6. The pharmaceutical composition of any one of claims 1-3, the antibody comprises a human IgG1 heavy chain constant domain and a human kappa light chain constant domain.

7. The pharmaceutical composition of claim 1, wherein the antibody is lonigutamab.

8. The pharmaceutical composition of any one of claims 1-7, wherein the pharmaceutical composition comprises at least 100 mg/ml, at least 125 mg/ml, at least 150 mg/ml, at least 175 mg/ml, at least 200 mg/ml, or at least 250 mg/ml of the anti-IGF-1R antibody.

9. The pharmaceutical composition of any one of claims 1-7, wherein the pharmaceutical composition comprises from 75 mg/ml to 300 mg/ml, from 100 mg/ml to 300 mg/ml, or from 125 mg/ml to 250 mg/ml of the anti-IGF-1R antibody.

10. The pharmaceutical composition of any one of claims 1-7, wherein the pharmaceutical composition comprises about 125 mg/ml, about 150 mg/ml, about 175 mg/ml, about 200 mg/ml, or about 250 mg/ml of the anti-IGF-1R antibody.

11. The pharmaceutical composition of any one of claims 1-10, wherein the pharmaceutical composition comprises about 20 mM, about 21 mM, about 22 mM, about 23 mM, about 24 mM, about 25 mM, about 26 mM, about 27 mM, about 28 mM, about 29 mM, or about 30 mM histidine.

12. The pharmaceutical composition of any one of claims 1-11, wherein the pharmaceutical composition comprises about 4%, 5%, or 6% D-sorbitol.

13. The pharmaceutical composition of any one of claims 1-12, wherein the pharmaceutical composition is at a pH of about 5.5, about 5.6, about 5.7, about 5.8, about 5.9, about 6.0, about 6.1, about 6.2, about 6.3, about 6.4, or about 6.5.

14. The pharmaceutical composition of any claims 1-13, wherein the osmolality of the pharmaceutical composition is within physiological osmolality range of 250-400 mOsm/kg.

15. The pharmaceutical composition of any one of claims 1-14, wherein the viscosity of the pharmaceutical composition is no more than 30 cP at 21° C.

16. The pharmaceutical composition of claim 15, wherein the viscosity of the pharmaceutical composition is no more than 15 cP at 21° C.

17. The pharmaceutical composition of claim 15, wherein the viscosity of the pharmaceutical composition is about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, about 23 cP, about 24 cP, about 25 cP, about 26 cP, about 27 cP, about 28 cP, about 29 cP, or about 30 cP at 21° C.

18. The pharmaceutical composition of any one of claims 1-17, wherein the pharmaceutical composition is stable for at least 8 weeks, at least 9 weeks, at least 10 weeks, at least 11 weeks, at least 12 weeks, at least 13 weeks, at least 14 weeks, at least 15 weeks, or at least 16 weeks.

19. The pharmaceutical composition of claim 18, wherein the pharmaceutical composition is stable at a temperature of from −70° C. to 40° C.

20. A method of treating thyroid eye disease (TED) comprising administering the pharmaceutical composition of any one of claims 1-19.

21. The method of claim 20, wherein the pharmaceutical composition is administered subcutaneously.

22. The method of claim 20, wherein the pharmaceutical composition is administered intramuscularly.

23. The method of any one of claims 20-22, wherein the pharmaceutical composition is administered in a delivery volume of no more than 2 ml.

24. The method of any one of claims 20-23, wherein the pharmaceutical composition is administered via a needle of a size of no bigger than 24G.

25. The method of any one of claims 20-24, wherein the pharmaceutical composition is administered with an injection force of no more than 12N.

26. The method of claim 25, wherein the pharmaceutical composition is administered with an injection force of about 4N, about 5N, about 6N, about 7N, about 8N, about 9N, about 10N, about 11N, or about 12N.

27. The method of any one of claims 20-26, wherein the method reduces the severity of the thyroid eye disease (TED).

28. The method of any one of claims 20-27, wherein the method reduces proptosis in an eye in a subject with thyroid eye disease (TED).

29. The method of claim 28, wherein proptosis is reduced by at least 2 mm.

30. The method of claim 28, wherein proptosis is reduced by at least 3 mm.

31. The method of claim 28, wherein proptosis is reduced by at least 4 mm.

32. The method of any one of claims 20-31, wherein the method reduces Clinical Activity Score (CAS) of thyroid eye disease (TED).

33. The method of claim 32, wherein the clinical activity score (CAS) is reduced by at least 2 points.

34. The method of claim 32, wherein the clinical activity score (CAS) is reduced to one (1).

35. The method of claim 32, wherein the clinical activity score (CAS) of the subject is reduced to zero (0).

36. The method of any one of claims 20-35, wherein the method improves the quality of life in the subject.

37. The method of claim 36, wherein the quality of life is measured by the Graves' Ophthalmopathy Quality of Life (GO-QoL) assessment.

38. The method of claim 36, wherein the quality of life is measured by the Visual Functioning or Appearance subscale thereof.

39. The method of claim 36, wherein the quality of life is measured by the European Group on Graves' orbitopathy (EUGOGO) guidelines.

40. The method of any one of claims 20-39, wherein the method reduces the severity of diplopia.

41. The method of claim 40, wherein the diplopia is constant diplopia.

42. The method of claim 40, wherein the diplopia is inconstant diplopia.

43. The method of claim 40, wherein the diplopia is intermittent diplopia.

44. An injector comprising the pharmaceutical composition of any one of claims 1-19.

45. The injector of claim 44, wherein the injector comprises a delivery volume of no more than 2 ml.

46. The injector of claim 44 or 45, wherein the injector comprises a needle of a size of no bigger than 24G.

47. The injector of any one of claims 44-46, wherein the injector is an automatic reusable fix dose Pen.

48. The injector of any one of claims 44-46, wherein the injector is an automatic reusable variable dose Pen.

49. The injector of any one of claims 44-46, wherein the injector is an automatic disposable fix dose autoinjector.

50. A method of treating thyroid eye disease (TED) comprising administering the pharmaceutical composition using the injector of any one of claims 44-49.

51. The method of claim 50, wherein the pharmaceutical composition is administered subcutaneously.

52. The method of claim 50, wherein the pharmaceutical composition is administered intramuscularly.

53. The method of any one of claims 50-52, wherein the pharmaceutical composition is administered with an injection force of no more than 12N.

54. The method of claim 53, wherein the pharmaceutical composition is administered with an injection force of about 4N, about 5N, about 6N, about 7N, about 8N, about 9N, about 10N, about 11N, or about 12N.

55. The pharmaceutical composition of any one of claims 1-19 for use in treating thyroid eye disease (TED).

56. The pharmaceutical composition for use according to claim 55, wherein the pharmaceutical composition is administered subcutaneously.

57. The pharmaceutical composition for use according to claim 55, wherein the pharmaceutical composition is administered intramuscularly.

58. The pharmaceutical composition for use according to any one of claims 55-57, wherein the pharmaceutical composition is administered in a delivery volume of no more than 2 mL.

59. The pharmaceutical composition for use according to any one of claims 55-58, wherein the pharmaceutical composition is administered via a needle of a size of no bigger than 24G.

60. The pharmaceutical composition for use according to any one of claims 55-59, wherein the pharmaceutical composition is administered with an injection force of no more than 12N.

61. The pharmaceutical composition for use according to claim 60, wherein the pharmaceutical composition is administered with an injection force of about 4N, about 5N, about 6N, about 7N, about 8N, about 9N, about 10N, about 11N, or about 1N.

62. An anti-IGF-1R antibody comprising:
(a) a heavy chain comprising a CDRH1 of SEQ ID NO: 1, a CDRH2 of SEQ ID NO: 2, a CDRH3 of SEQ ID NO: 3, and a charged amino acid on its c-terminus; and
(b) a light chain comprising a CDRL1 of SEQ ID NO: 4, a CDRL2 of SEQ ID NO: 5, and a CDRL3 of SEQ ID NO: 6.

63. The anti-IGF-IR antibody of claim 62, wherein the heavy chain comprises a variable domain comprising an amino acid sequence of SEQ ID NO: 7.

64. The anti-IGF-1R antibody of claim 62 or 63, wherein the light chain comprises a variable domain comprising an amino acid sequence of SEQ ID NO: 8.

65. The anti-IGF-1R antibody of any one of claims 62-64, wherein the charged amino acid is a positively charged amino acid.

66. The anti-IGF-1R antibody of claim 65, wherein the positively charged amino acid is a lysine, a histidine, or an arginine.

67. The anti-IGF-IR antibody of claim 66, wherein the positively charged amino acid is a lysine.

68. The anti-IGF-1R antibody of claim 65, wherein the heavy chain comprises a sequence of SEQ ID NO: 11.

69. The anti-IGF-IR antibody of claim 65, wherein the heavy chain comprises a sequence of SEQ ID NO: 12.

70. The anti-IGF-IR antibody of claim 65, wherein the heavy chain comprises a sequence of SEQ ID NO: 13.

71. The anti-IGF-1R antibody of any one of claims 62-64, wherein the charged amino acid is a negatively charged amino acid.

72. The anti-IGF-IR antibody of claim 71, wherein the positively charged amino acid is a aspartic acid or glutamic acid.

73. The anti-IGF-1R antibody of claim 71, wherein the heavy chain comprises a sequence of SEQ ID NO: 14.

74. The anti-IGF-1R antibody of claim 71, wherein the heavy chain comprises a sequence of SEQ ID NO: 15.

75. A pharmaceutical composition comprising an antibody of any one of claims 62-74.

76. A method of treating thyroid eye disease (TED) comprising administering the pharmaceutical composition of claim 75.

77. The method of claim 76, wherein the pharmaceutical composition is administered subcutaneously.

78. The method of claim 76, wherein the pharmaceutical composition is administered intramuscularly.

79. The pharmaceutical composition of claim 75 for use in treating thyroid eye disease (TED).

80. The pharmaceutical composition for use according to claim 79, wherein the pharmaceutical composition is administered subcutaneously.

81. The pharmaceutical composition for use according to claim 79, wherein the pharmaceutical composition is administered intramuscularly.

82. A method of treatment of thyroid eye disease (TED) comprising administering to a patient a pharmaceutical composition comprising a therapeutically effective dose of lonigutamab as an injection.

83. The method of claim 82, wherein the injection is an intravenous infusion.

84. The method of claim 82, wherein the injection is administered subcutaneously.

85. The method of claim 83, wherein lonigutamab is administered as an intravenous infusion at a dose of about 0.1 mg/kg.

86. The method of claim 83, wherein lonigutamab is administered as an intravenous infusion at a dose of about 0.3 mg/kg.

87. The method of claim 83, wherein lonigutamab is administered as an intravenous infusion at a dose of about 1.0 mg/kg.

88. The method of claim 83, wherein lonigutamab is administered as an intravenous infusion at a dose of about 3.0 mg/kg.

89. The method of claim 84, wherein lonigutamab is administered as a subcutaneous injection comprising 20 mg lonigutamab.

90. The method of claim 84, wherein lonigutamab is administered as a subcutaneous injection comprising 40 mg lonigutamab.

91. The method of claim 84, wherein lonigutamab is administered as a subcutaneous injection comprising 125 mg lonigutamab.

92. The method of claim 84, wherein lonigutamab is administered as a subcutaneous injection comprising 250 mg lonigutamab.

93. The method of claim 82, wherein the administration of a therapeutically effective dose of lonigutamab results in serum concentration of about 3 μg/mL or higher.

94. The method of claim 83, wherein the intravenous administration was conducted for a duration of up to about 60 minutes.

95. The method of claim 82, wherein the administration of lonigutamab have minimal adverse effects.

96. The method of claim 82, wherein the administration of pharmaceutical compositions comprising lonigutamab achieve IGF-1R occupancy of 95% or higher.

97. The method of claim 96, wherein the IGF-1R occupancy of 95% or higher may be achieved 1 hour after administration of the pharmaceutical composition.

98. The method of claim 96, wherein IGF-1R occupancy of 90% or higher is maintained for a duration of at least 28 days after administration of the pharmaceutical composition comprising lonigutamab.

99. A method of treatment of thyroid eye disease (TED) comprising subcutaneous administration of a pharmaceutical composition comprising from about 10 mg to about 300 mg lonigutamab.

100. The method of claim 99, wherein the pharmaceutical composition comprises about 20 mg lonigutamab.

101. The method of claim 99, wherein the pharmaceutical composition comprises about 125 mg lonigutamab.

102. The method of claim 99, wherein the pharmaceutical composition comprises about 250 mg lonigutamab.

103. The method of claim 99, wherein the pharmaceutical composition is administered once weekly.

104. The method of claim 99, wherein the pharmaceutical composition is administered once every two weeks.

105. The method of claim 99, wherein the pharmaceutical composition is administered once every three weeks.

106. The method of claim 99, wherein the pharmaceutical composition is administered once every four (4) weeks.

107. The method of claim 100, wherein the pharmaceutical composition is administered at day 1 and day 21.

108. The method of claim 101, wherein the pharmaceutical composition is administered at day 1 and day 21.

109. The method of claim 102, wherein the pharmaceutical composition is administered at day 1 and day 21.
